(19) United States
(12) Patent Application Publication
(10) Pub. No.: US 2015/0165091 A1
(43) Pub. Date: Jun. 18, 2015

(54) CONTROLLING EXTRACELLULAR MATRIX PROTEIN MICROSTRUCTURE WITH ULTRASOUND

(71) Applicant: UNIVERSITY OF ROCHESTER, Rochester, NY (US)

(72) Inventors: Diane Dalecki, Rochester, NY (US); Denise Hocking, Rochester, NY (US); Kelley Garvin, Rochester, NY (US)

(21) Appl. No.: 14/389,283

(22) PCT Filed: Mar. 14, 2013
(86) PCT No.: PCT/US13/31552

§ 371 Date: Sep. 29, 2014

Related U.S. Application Data
(60) Provisional application No. 61/617,986, filed on Mar. 30, 2012.

(51) Int. Cl. A61L 27/24; A61L 27/52; A61L 27/36; A61L 27/54; A61L 27/38

(52) U.S. Cl. CPC A61L 27/24; A61L 27/54; A61L 27/3826; A61L 27/3633; A61L 27/52; A61L 2430/00; A61L 2430/40; A61L 2400/18

(57) ABSTRACT

The present invention is directed to methods of controlling extracellular matrix protein microstructure in a biological composition using ultrasound technology. The invention is further directed to three-dimensional monolithic tissue scaffolds having spatially defined regions of varying extracellular matrix protein microstructure and engineered tissue constructs comprising the same.

This application claims the benefit of U.S. Provisional Patent Application Ser. No. 61/617,986, filed Mar. 30, 2012, which is hereby incorporated by reference in its entirety.

This invention was made with government support under grant numbers R01EB008368 and R01EB008996 awarded by the National Institutes of Health. The government has certain rights in this invention.

### FIELD OF THE INVENTION

The present invention is directed to methods of controlling extracellular matrix protein microstructure in a biological composition. The present invention is also directed to tissue scaffolds and tissue constructs having defined extracellular matrix protein microstructures.

### BACKGROUND OF THE INVENTION

Since the early 1960s, tissue transplantation has been a highly successful therapy for end-stage organ failure (Nasseri et al., “Tissue Engineering: An Evolving 21st-Century Science to Provide Biologic Replacement for Reconstruction and Transplantation,” Surgery 130:781-784 (2001); Atala, A., “Engineering Organs,” Curr. Opin. Biotechnol. 20:575-592 (2009)). However, present demand for donor organs far exceeds the available supply. Currently, over 115,000 patients in the United States are awaiting organ transplantation (US Department of Health and Human Services). By developing methods for regenerating diseased or injured tissues and organs, tissue engineering seeks to provide an alternative supply of tissues and organs to balance supply and demand. Engineered tissues could also provide alternatives to traditional animal models used currently for product and procedure testing, toxicology screening, drug discovery, and biological and chemical warfare detection. One of the most common approaches for producing engineered tissue involves implanting cells within biologically- or synthetically-derived, three-dimensional scaffolds (Butler et al., “Functional Tissue Engineering: The Role of Biomechanics,” J. Biomech. Eng. 122:570-575 (2000); Stock & Vacanti, “Tissue Engineering: Current State and Prospects,” Annu. Rev. Med. 52:443-451 (2001)). The current lack of available tissue analogs reflects an inability to create three-dimensional scaffolds that have both biological activity and adequate mechanical strength.

The formation of new tissue depends upon dynamic interactions between cells and their surrounding extracellular matrix (Frantz et al., “The Extracellular Matrix at a Glance,” J. Cell. Sci. 123:4195-4200 (2010)). The extracellular matrix is a complex network of interconnected proteins and polysaccharides that imparts structure and mechanical stability to tissues, and provides cell adhesion sites, migration pathways, and proliferation signals to cells (Frantz et al., “The Extracellular Matrix at a Glance,” J. Cell. Sci. 123:4195-4200 (2010)). In turn, the precise composition and organization of the extracellular matrix contribute to the mechanical and permeability properties of tissues and organs (Frantz et al., “The Extracellular Matrix at a Glance,” J. Cell. Sci. 123:4195-4200 (2010)). Collagen is the primary fibrous component of the extracellular matrix, where it plays a central role in embryonic development and tissue repair (Rozario & DeSimone, “The Extracellular Matrix in Development and Morphogenesis: A Dynamic View,” Dev. Biol. 341:126-140 (2009)). Twenty-eight different types of collagen exist, which are categorized by structure and organization into several different families (Kadler et al., “Collagens at a Glance,” J. Cell. Sci. 120:1955-1958 (2007)). Approximately 90% of all collagens belong to the family of fibril-forming collagens (Kadler et al., “Collagens at a Glance,” J. Cell. Sci. 120:1955-1958 (2007)). Of the fibrillar collagens, type I collagen is the most abundant. It is the principal component of bone, tendons, skin, ligaments, as well as cornea, and is found in most interstitial connective tissues, where it provides tensile strength to tissues, regulates cell adhesion, and facilitates cell migration (Kadler et al., “Collagens at a Glance,” J. Cell. Sci. 120:1955-1958 (2007)). Clinically, type I collagen is used widely in wound dressings, for skin substitutes, and as a natural component of biomaterials in tissue engineering and regenerative medicine applications (Lee et al., “Biomedical Applications of Collagen,” Int. J. Pharm. 221:1-22 (2001); Cen et al., “Collagen Tissue Engineering: Development of Novel Biomaterials and Applications,” Pediatr. Res. 63:492-496 (2008)). Collagen has numerous advantages as a biomaterial, including low toxicity and antigenicity, biodegradability, and high abundance ((Lee et al., “Biomedical Applications of Collagen,” Int. J. Pharm. 221:1-22 (2001)).

The incredible diversity of the functional properties of type I collagen arises from variations in the micro- and macromolecular structure of polymerized collagen fibers. Type I collagen molecules assemble as right-handed triple helices that bundle together in a staggered fashion to form long thin fibrils with diameters of ˜25-500 nm (Gelse et al., “Collagens—Structure, Function, and Biosynthesis,” Adv. Drug Deliv. Rev. 55:1531-1546 (2003); Shoulders & Raines, “Collagen Structure and Stability,” Annu. Rev. Biochem. 78:929-958 (2009)). In vitro, type I collagen fibers can form spontaneously through a self-assembly process. The physical properties of self-assembled collagen fibers, including fibril density, thickness, and alignment, are influenced by several factors, namely collagen concentration, temperature, pH, ionic strength, and applied mechanical forces (Roeder et al., “Tensile Mechanical Properties of Three-Dimensional Type I Collagen Extracellular Matrices With Varied Microstructure,” J. Biomech. Eng. 124:214-222 (2002); Sander & Barocas, “Biomimetic Collagen Tissues: Collagenous Tissue Engineering and Other Applications” in P. Fratzl, ed., Collagen Structure and Mechanics, Springer, New York, N.Y., pp. 475-504 (2008)). Differences in the physical parameters of fibrillar collagens, such as stiffness, fiber orientation, and ligand presentation, affect cell and tissue function (Gelse et al., “Collagens—Structure, Function, and Biosynthesis,” Adv. Drug Deliv. Rev. 55:1531-1546 (2003); Shoulders & Raines, “Collagen Structure and Stability,” Annu. Rev. Biochem. 78:929-958 (2009)). As such, controlling the structure of type I collagen would provide a means to regulate the mechanical properties of biomaterials and control cellular responses in engineered tissues.

The present invention is directed to overcoming these and other deficiencies in the art.

### SUMMARY OF THE INVENTION

A first aspect of the present invention is directed to a method of controlling extracellular matrix protein microstructure in a biological composition. This method involves providing a biological composition comprising one or more soluble unpolymerized extracellular matrix proteins and adjusting the conditions of the biological composition to effect extracellular matrix protein polymerization. The method further involves exposing the biological composition to one or more ultrasound wave fields during extracellular matrix protein polymerization under conditions effective to produce two or more spatially defined regions within the biological composition that comprise different extracellular matrix protein microstructures.

A second aspect of the present invention is directed to a method of controlling extracellular matrix protein microstructure in an acellular biological composition. This method involves providing an acellular biological composition comprising one or more soluble unpolymerized extracellular matrix proteins and adjusting the conditions of the acellular biological composition to effect extracellular matrix protein polymerization. The method further involves exposing the acellular biological composition to one or more ultrasound wave fields during extracellular matrix protein polymerization under conditions effective to control the extracellular matrix protein microstructure within the acellular biological composition.

A third aspect of the present invention is directed to a method of controlling extracellular matrix protein microstructure in a biological composition. This method involves providing a biological composition comprising one or more soluble unpolymerized extracellular matrix proteins and adjusting the conditions of the biological composition to effect extracellular matrix protein polymerization. The method further involves exposing the biological composition to one or more ultrasound traveling wave fields during extracellular matrix protein polymerization under conditions effective to control the extracellular matrix protein microstructure within the biological composition.

Another aspect of the present invention is directed to a monolithic tissue scaffold comprising two or more spatially defined, non-overlapping regions, wherein at least two of the regions comprise different extracellular matrix protein microstructures.

The present invention allows for non-invasive control of extracellular matrix protein microstructure during the fabrication of three-dimensional engineered tissues. The structure and organization of the extracellular matrix regulates key cellular functions, such as survival, growth, and migration. In addition, the microstructure of the extracellular matrix proteins within tissue constructs influences the mechanical strength of the tissue. Thus, the ability to non-invasively control extracellular matrix microstructure within three-dimensional engineered tissue as described herein provides the opportunity to produce a wide variety of artificial tissues that each more closely mimic their in vivo tissue and organ counterparts.

### BRIEF DESCRIPTION OF THE DRAWINGS

FIGS. 1A-1B schematically illustrate the experimental design for ultrasound exposures. FIG. 1A is a schematic of the experimental set-up used for ultrasound standing wave field exposures. The acoustic source, either a 1-MHz, 2.5-cm diameter or an 8.3-MHz, 0.64-cm diameter unfocused piezoceramic transducer, was situated at the bottom of a plastic exposure tank filled with degassed, deionized water. Well bottoms were placed near the air-water interface a distance, d (12.2 cm for the 1 MHz source and 6.7 cm for the 8.3 MHz source) from the transducer. FIG. 1B is a schematic of the experimental set-up used for ultrasound traveling wave field exposures. The acoustic source was a 1-MHz, 2.5-cm diameter unfocused piezoceramic transducer. The three-axis positioner was used to place the front face of the sample holder a distance, d, of 12.2 cm from the transducer. A rubber absorber was placed behind the cuvette to reduce reflections

FIGS. 2A-2B show that ultrasound standing wave field exposure alters collagen fiber microstructure within three-dimensional collagen gels to thin, short, dense fiber networks. Solutions of type I collagen were exposed during the polymerization process to 1-MHz CW ultrasound using various peak positive pressure amplitudes and Iₛₚₜₐ values. Resultant collagen gels were analyzed for collagen fiber structure using second-harmonic generation microscopy. FIG. 2A shows representative images of collagen fibers collected at the antinode (top panel) and node (bottom panel) imaging depths are shown. Scale bar, 50 μm. In FIG. 2B, the images of FIG. 2A were analyzed to quantify collagen fiber density. Data for antinode (left panel) and node (right panel) imaging depths are presented as average fold difference in fiber density±SEM normalized to sham average fiber density values (n=3; *p<0.05).

FIGS. 3A-3F show that ultrasound exposure alters collagen fiber structure in a non-transient manner and the ultrasound-induced change in collagen microstructure is only achieved during the polymerization process. Unpolymerized solutions of type I collagen were exposed during the polymerization process to 1-MHz CW ultrasound using an Iₛₚₜₐ of 2.4 W/cm² (0.3 MPa). Resultant collagen gels were incubated at 37° C. for either 20 min (FIGS. 3A and 3B) or 20 hr (FIGS. 3C and 3D), then fixed and imaged using second-harmonic generation microscopy. In FIGS. 3E and 3F, polymerized type I collagen gels were exposed to ultrasound and processed for imaging as above. Representative images of collagen fibers collected at the antinode imaging depth are shown (n=3). Scale bar, 50 μm.

FIGS. 4A-4B show that temporal average intensity of the sound field correlates with changes in collagen fiber structure. Unpolymerized solutions of type I collagen were exposed to a 1-MHz pulsed or CW ultrasound field (20 μs pulse duration with duty cycles from left to right of in FIG. 4A, 0, 0.12, 0.24, 0.5, and 1, and in FIG. 4B, 0, 1, 0.5, 0.24, and 0.12) using a constant ultrasound standing wave field peak positive pressure amplitude of 0.3 MPa and various Iₛₚₜₐ (FIG. 4A), or a constant Iₛₚₜₐ of 0.28 W/cm² and various ultrasound standing wave field peak positive pressure amplitudes (FIG. 4B). Resultant collagen gels were fixed and analyzed for collagen fiber structure using second-harmonic generation microscopy. Representative images of collagen fibers collected at the antinode imaging depth are shown (n=3). Scale bar, 50 μm.

FIGS. 5A-5B show the effects of ultrasound-induced heating of collagen solutions. Solutions of type I collagen were exposed during the polymerization process to 1-MHz, CW ultrasound using an Iₛₚₜₐ of 2.4 W/cm². In FIG. 5A, temperatures of ultrasound and sham-exposed collagen gels are plotted as mean±SEM (n=3). FIG. 5B shows representative images of collagen fibers collected using second-harmonic generation microscopy (n=3). Scale bar, 50 μm.

FIG. 6 demonstrates that ultrasound exposure of collagen affects fiber microstructure. Solutions of type I collagen were exposed to a 1-MHz pulsed or CW ultrasound field (20 μs pulse duration with duty cycles of 0, 1, 0.5, 0.24, and 0.12 from left to right) using a constant Iₛₚₜₐ of 2.4 W/cm² and various ultrasound standing wave field peak positive pressure amplitudes. Representative images of collagen fibers collected using second-harmonic generation microscopy at a pressure antinode imaging depth (n=3). Scale bar, 50 μm

FIGS. 7A-7B shows tests conducted with and without elastomer mold to further investigate the thermal effects of ultrasound-induced heating on collagen fiber microstructure. Solutions of type I collagen were exposed to 1-MHz CW ultrasound using an Iₛₚₜₐ of 2.4 W/cm². Samples were either contained within 1-cm (elastomer mold present) or 2.5-cm diameter (elastomer mold absent) wells. In FIG. 7A, sample temperatures are plotted as mean±SEM (n=3). FIG. 7B shows representative images of collagen fibers collected using second-harmonic generation microscopy at the antinodal imaging depth (n=3). Scale bar, 50 μm.

FIGS. 8A-8B show that ultrasound traveling wave fields produce changes in collagen microstructure. Solutions of type I collagen were exposed to 1-MHz ultrasound using either an ultrasound standing wave field or traveling wave field experimental set-up. In FIG. 8A, temperature measurements were obtained under various exposure conditions and are plotted as mean±SEM (n=3). FIG. 8B shows representative images of collagen fibers collected using second-harmonic generation microscopy (n=3). Scale bar, 50 μm.

FIGS. 9A-9E show that spatial patterning of distinct collagen fiber structures within a single collagen gel is achieved using high frequency ultrasound exposure. FIG. 9A is a schematic of the method utilized to locally control collagen fiber microstructure. A high frequency, 8.3-MHz acoustic source, with a narrow beam width (−6 dB=0.6 cm) was directed at the center of a collagen sample. In FIG. 9B, solutions of type I collagen were exposed to 8.3-MHz CW ultrasound using an ultrasound standing wave field with an Iₛₚₜₐ of 30 W/cm². Samples were contained within 4-cm diameter wells of BioFlex® culture plates. Temperatures were measured at the gel center and periphery (outside the −6 dB beam width) using two thermocouples. Temperatures are plotted as mean±SEM (n=3). FIG. 9C shows representative second-harmonic generation microscopy images of collagen fibers collected at the center and periphery of sham-exposed samples and samples exposed to 30 W/cm² Iₛₚₜₐ (n=3). Scale bar, 50 μm. FIG. 9D shows representative scanning electron microscopy images of collagen fibers from sham and ultrasound-exposed (30 W/cm²) samples (n=3). Scale bar, 1 μm. In FIG. 9E, scanning electron microscopy images were used to quantify collagen fiber diameters. Data are presented as histograms to display the frequency of occurrence of fiber diameters as a percentage of the total number of fibers measured in both sham- and ultrasound-exposed collagen gel centers (n=3).

FIG. 10 depicts the cellular response to ultrasound-exposed collagen gels. Solutions of type I collagen were exposed to 8.3-MHz CW ultrasound using an ultrasound standing wave field Iₛₚₜₐ of 30 W/cm². Fibronectin-null mouse embryonic myofibroblasts were seeded onto resultant collagen gels, and cell location and morphology were monitored over time using phase-contrast microscopy. Shown are representative images collected at the gel center and gel periphery of ultrasound-exposed and sham-exposed samples 1 hr and 1 day post-cell seeding. Arrow denotes area of the collagen substrate devoid of cells (n=3). Scale bar, 200 μm.

FIG. 11 shows cell-mediated reorganization of ultrasound-exposed collagen fibers. Solutions of type I collagen were exposed to 8.3-MHz, CW ultrasound using an ultrasound standing wave field Iₛₚₜₐ of 30 W/cm². Fibronectin-null mouse embryonic myofibroblasts were seeded onto resultant collagen gels and samples were fixed 1 or 28 days post-cell seeding. Samples were analyzed using two-photon and second-harmonic generation microscopy. Representative merged images show reorganization of ultrasound-exposed collagen fibers at gel centers, first into aligned fiber bundles between cellular aggregates at day 1 (arrows), and into densely packed sheets at 28 days (arrowheads) (n=3). Cells, green; collagen fibers, red. Scale bar, 50 μm.

FIG. 12 shows that cellular responses to collagen gels exposed to ultrasound during polymerization differ from those of collagen gels exposed to bulk heating during polymerization. Mouse embryonic myofibroblasts were seeded onto collagen gels fabricated at 37° C. in the absence of ultrasound exposure. Images were obtained by phase contrast microscopy on days 1 and 28 post-seeding at both the gel center and periphery. Scale bar is 200 μm.

### DETAILED DESCRIPTION OF THE INVENTION

This invention relates generally to methods of controlling or modifying the extracellular matrix microstructure within biological compositions, such as three-dimensional tissue scaffolds and tissue constructs, such that the resulting compositions more closely mimic the structural complexity of the in vivo tissue environment. Accordingly, a first aspect of the present invention is directed to a method of controlling extracellular matrix protein microstructure in a biological composition. This method involves providing a biological composition comprising one or more soluble unpolymerized extracellular matrix proteins and adjusting the conditions of the biological composition to effect extracellular matrix protein polymerization. The method further involves exposing the biological composition to one or more ultrasound wave fields during extracellular matrix protein polymerization under conditions effective to produce two or more spatially defined regions within the biological composition that comprise different extracellular matrix protein microstructures.

A second aspect of the present invention is directed to a method of controlling extracellular matrix protein microstructure in an acellular biological composition. This method involves providing an acellular biological composition comprising one or more soluble unpolymerized extracellular matrix proteins and adjusting the conditions of the acellular biological composition to effect extracellular matrix protein polymerization. The method further involves exposing the acellular biological composition to one or more ultrasound wave fields during extracellular matrix protein polymerization under conditions effective to control the extracellular matrix protein microstructure within the acellular biological composition.

A third aspect of the present invention is directed to a method of controlling extracellular matrix protein microstructure in a biological composition. This method involves providing a biological composition comprising one or more soluble unpolymerized extracellular matrix proteins and adjusting the conditions of the biological composition to effect extracellular matrix protein polymerization. The method further involves exposing the biological composition to one or more ultrasound traveling wave fields during extracellular matrix protein polymerization under conditions effective to control the extracellular matrix protein microstructure within the biological composition.

As used herein, a “biological composition” encompasses any biological composition comprising as least one soluble unpolymerized extracellular matrix (ECM) protein, including for example, but without limitation, collagens, fibrin, fibronectin, laminin, elastin, fibrinogen, hyaluronin, and vitronectin. In one embodiment of the invention, the biological composition comprises one or more collagens, i.e., one or more of the twenty-eight different known types of collagen. In a preferred embodiment of the invention, the biological composition comprises at least collagen I, collagen II, collagen III, collagen IV, or collagen V or any combination thereof. The ECM protein or proteins are present in the composition at a concentration sufficient to form a three-dimensional structure upon polymerization. In one embodiment of the present invention, the biological composition is an acellular biological composition, i.e., the composition itself does not contain living cells. The resulting three-dimensional structure formed upon polymerization of the one or more ECM proteins in a biological composition, including an acellular biological composition, is suitable for use as a tissue scaffold, i.e., a structure or substrate suitable for supporting the adhesion, growth, proliferation, migration, etc. of cells ex vivo and in vivo, in an engineered tissue construct.

In accordance with its use as a tissue scaffold, the biological composition of the present invention can further contain other components of the ECM, such as, for example, glycosaminoglycans (e.g., heparin sulfate, chondroitin sulfate, keratan sulfate, and hyaluronic acid), related proteoglycans (e.g., aggrecan, betaglycan, decorin, perlecan, syndecan), glycoproteins (e.g., fibulin-1, tenascin-C, lectins, selectins), chemotactic agents, or growth factors, for example, cytokines, eicosanoids, or differentiation factors. The biological composition can also contain biologically active peptides or peptidomimetics of extracellular proteins, e.g., collagens, fibrin, fibronectin, laminin, elastin, fibrinogen, and vitronectin, growth factors, or differentiation factors. Exemplary biological compositions of the present invention which comprise one or more of the aforementioned components include, without limitation, hydrogels and matrigels.

The biological composition may further contain one or more biological support materials, such as, e.g., filaments, meshes, foams, gels, plastics, ceramics, and acellularized extra-cellular matrix material. The biological support material preferably consists of a biocompatible material, e.g., a biocompatible polymer having properties or incorporating modifications that are conducive to cell adherence and/or growth. In one embodiment, the support material is a porous polymer as described in U.S. Pat. No. 6,103,255 to Levene, which is hereby incorporated by reference in its entirety. In another embodiment, the support material is biodegradable or bioerodable, including those materials that hydrolyze slowly under physiological conditions. Suitable materials, include synthetic polymeric materials such as polyesters, polyorthoesters, polylactic acid, polyglycolic acid, polycaprolactone, or polyanhydrides, including polymers or copolymers of glycolic acid, lactic acid, or sebacic acid. Substrates comprising proteinaceous polymers are also suitable for use in the methods of the present invention.

Suitable biological compositions used in the methods of the present invention may further contain one or more particles that are also responsive to the ultrasound wave field exposure. In one embodiment of the invention, the one or more particles become spatially organized within the biological composition in response to ultrasound exposure as described in WO10/135,044 to Dalecki et al., and Garvin et al., “Controlling the Spatial Organization of Cells and Extracellular Matrix Proteins in Engineered Tissues Using Ultrasound Standing Wave Fields,” Ultrasound Med. Biol. 36(100):1919-32 (2010), which are hereby incorporated by reference in their entirety. Particles responsive to ultrasound wave fields include, without limitation, nanoparticles, microparticles, microbubbles, and cells. In a preferred embodiment of the present invention, these particles contain or have bound thereto biologically active peptides, proteins, or peptide or protein mimetics. Suitable peptides, proteins, and protein mimetics include for example growth factors (e.g., VEGF or FGF), adhesion proteins, extracellular matrix proteins (e.g., fibronectin, recombinant fibronectin fragments, or vitronectin), and/or angiogenic factors (e.g., FGF, bFGF, acid FGF (aFGF), FGF-2, FGF-4, EGF, PDGF, TGF-beta, angiopoietin-1, angiopoietin-2, placental growth factor (PlGF), VEGF, and PMA (phorbol 12-myristate 13-acetate). In one embodiment of the present invention, the biologically active peptide, protein, or protein mimetic is a biologically active fibronectin peptide, protein, or protein mimetic.

In another embodiment of the present invention, the one or more particles responsive to ultrasound exposure are cells. Spatial organization of cells within a three-dimensional biological composition facilitates proper cell function and growth. For example, the spatial organization of endothelial cells within a biological composition facilitates neovessel formation and results in production of a vascularized three-dimensional tissue construct as described in WO10/135,044 to Dalecki et al., which is hereby incorporated by reference in its entirety.

In another embodiment of the present invention the biological compositions comprise both cells and particles that are responsive to ultrasound exposure mediated spatial organization. In accordance with this embodiment, exposure of the cells and particles (e.g. proteins bound to cells) to an ultrasound standing wave field can be used to co-localize the particles with the cells. Co-localization of the particles and cells in combination with the controlled extracellular matrix protein microstructure provides cells with the necessary and specific signals and growth factors that help mediate cell growth, proliferation, or differentiation in a controlled manner. In another embodiment of the present invention, exposure of the cells and particles (e.g. microbubbles) to an ultrasound wave field can be used to produce a staggered arrangement of the particles and cells within the biological composition. This staggered arrangement of particles and cells will generate a concentration gradient of growth factors or matrix proteins contained in the particles between the spatially organized cells. The concentration gradients in combination with controlled extracellular matrix protein microstructure will enhance phenotypic differentiation of the spatially organized cells (e.g., enhance neovessel formation of endothelial cell bands).

Once the desired biological composition has been established, the conditions of the biological composition are adjusted to effectuate polymerization of the one or more unpolymerized ECM proteins in the composition. Polymerization conditions for ECM proteins are generally a function of temperature and pH, and optimal conditions are at or near physiological conditions for most ECM proteins. For example, collagen polymerization occurs at a pH range of 6.5-8.5 and temperature range of 18-37° C. Once the ECM proteins of the biological composition begin the polymerization process, the biological composition is exposed to one or more ultrasound wave fields to control extracellular matrix protein microstructure within and throughout the composition either uniformly or in a site-specific manner.

Ultrasound is a form of mechanical energy that travels through a medium of propagation as an acoustic pressure wave at frequencies above 20 kHz. It is used widely in the medical field as both a diagnostic and therapeutic tool (Duck et al., “Ultrasound In Medicine,” Philadelphia: Institute of Physics Publishing (1998), which is hereby incorporated by reference in its entirety). Ultrasound can interact with biological tissues and compositions through thermal or mechanical mechanisms (Dalecki D., “Mechanical Bioeffects of Ultrasound,” Annu. Rev. Biomed. Eng. 6:229-248 (2004), which is hereby incorporated by reference in its entirety). Mechanical interactions, associated with the generation of ultrasound-induced mechanical forces in the medium of propagation, can produce ultrasounds bioeffects (Dalecki D., “Mechanical Bioeffects of Ultrasound,” Annu. Rev. Biomed. Eng. 6:229-248 (2004), which is hereby incorporated by reference in its entirety). In accordance with the methods of the present invention, ultrasound mediated control of extracellular matrix protein “microstructure” encompasses controlled changes to extracellular matrix protein fiber size (i.e., width and length), fiber number, fiber organization, fiber density, or any combination thereof within the biological composition. For example, as described in the Examples herein, applicants have shown that exposure of a biological composition containing collagen I to an ultrasound wave field during polymerization changes collagen fiber thickness, length, density, and fiber number. More specifically, ultrasound exposure induces the formation of short, thin, densely packed collagen fibers in a composition compared to the long, thick, more loosely packed collagen fibers of the non-exposed biological composition.

Unpolymerized collagen molecules typically consist of a homogenous collection of thin rod shaped molecules of ˜1.5 nm wide and 300 nm long (Sung et al., “Control of 3-Dimensional Collagen Matrix Polymerization for Reproducible Human Mammary Fibroblast Cell Culture in Microfluidic Devices,” Biomaterials 30:4833-4841 (2009), which is hereby incorporated by reference in its entirety). Polymerization of the collagen fibers at or around room temperature, i.e., 22° C., in the absence of ultrasound exposure generates collagen fibers having a width of about 200 nm to 1 μm and a length of about 8 to 20 nm. In contrast, collagen fiber width in compositions exposed to ultrasound is significantly decreased, ranging from about 20 nm to 200 nm wide, depending on the ultrasound exposure protocol. In addition, ultrasound exposure significantly alters fiber length, e.g., collagen fiber length following ultrasound exposure ranges from about 2 to 10 nm.

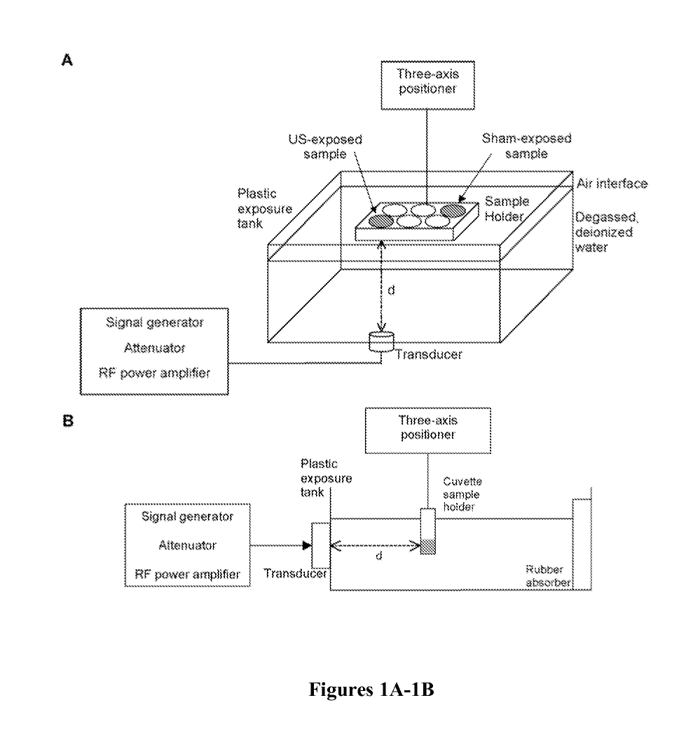

Prior to ultrasound wave field exposure, the biological composition is placed in a suitable ultrasound exposure chamber, such as the one described herein and shown in FIG. 1A for standing wave field exposures and FIG. 1B for traveling wave field exposures. The biological composition is positioned in the exposure tank via a sample holder. The sample holder aligns the biological composition with the ultrasound wave beam to facilitate direct exposure. The exposure chamber can further include a means for controlling the sample/air interface (e.g., a rubber absorber) to allow for ultrasound standing or traveling wave fields to be generated in the same exposure chamber.

The parameters of ultrasound wave field exposure of a biological composition will vary depending on the extracellular matrix protein composition and the desired endpoint, e.g. site-specific variation in microstructure throughout the composition vs. uniform microstructure throughout the compositions, and should be optimized for each biological composition to produce optimal results. These parameters include, for example, acoustic pressure amplitude, spatial peak temporal average intensity, frequency, and exposure duration as described in more detail below. Methods for optimizing these ultrasound exposure parameters are described herein.

As demonstrated herein, control of extracellular matrix protein microstructure can be achieved using either ultrasound standing wave fields, traveling wave fields, or a combination of both standing wave and traveling wave fields. Exposure to one beam of ultrasound is sufficient for inducing changes to the ECM protein microstructure. Alternatively, exposure of the composition to multiple intersecting or non-intersecting beams of ultrasound can be used to site-specifically control ECM protein microstructure formation. Various transducer geometries and set-ups can be utilized to achieve the desired spatial arrangement or organization of the ECM microstructures, e.g., cylinders, tubes, grids, ellipses, circles, rectangles, squares, triangles, spheres, rings, disks, plates/planes. The ultrasound field exposure may involve exposure of the biological composition to a continuous wave signal or a pulsating wave signal. If a pulsating wave signal exposure is employed, the appropriate pulse frequency and duration must be optimized. Exemplary conditions for standing and traveling pulsating wave signal exposure are disclosed herein in the Examples.

In accordance with this aspect of the present invention, exposure to the ultrasound field is carried out at a pressure amplitude and a spatial peak, temporal average intensity (Iₛₚₜₐ) that is optimal for mediating the extracellular matrix protein microstructure. In one embodiment of the present invention, the biological composition is exposed to ultrasound pressure amplitude of at least 0.1 MPa, preferably between 0.1 MPa and 1 MPa. Likewise, the biological composition is exposed to a spatial peak temporal average intensity of at least 0.1 W/cm², preferably between 0.1 W/cm² and 100 W/cm². The ultrasound frequency is also optimized to ensure exposure at a frequency that promotes the desired changes in ECM microstructure. In one embodiment, exposure of the biological composition to an ultrasound energy source, i.e., a standing wave or traveling wave field, is at a frequency of from about 0.1 MHz to about 75 MHz. More preferably the exposure to an ultrasound energy source is at a frequency of from about 0.5 MHz to about 25 MHz. In many embodiments, the optimal ultrasound frequency is dependent on the exposure geometry that is employed. For example, if it is desirable to achieve one uniform ECM protein microstructure throughout the biological composition, a lower frequency, e.g., at or around 1 MHz, may be optimal. Alternatively, if it is desirable to achieve site-specific ECM protein microstructure, a higher frequency, e.g., at or around 8 MHz may be optimal.

The duration of ultrasound exposure will vary depending on the extracellular matrix protein composition of the biological composition and the extent to which the microstructure of the composition is to be controlled. In one embodiment, ultrasound exposure is carried out for a period of about 10 seconds to about 60 minutes. In another embodiment the exposure is for a period of about 60 seconds to about 20 minutes. In another embodiment, the ultrasound is applied for about 15 minutes. Exposure of the biological composition to the ultrasound wave field may be repeated one or more times.

As noted above, in some embodiments of the present invention ECM protein microstructure is controlled in a site-specific manner to achieve spatial patterning of the ECM protein microstructure throughout the biological composition. In other words, the resulting biological composition may have two or more spatially defined regions that comprise different extracellular matrix protein microstructures. In some embodiments of the present invention, the different regions of extracellular microstructure are overlapping, while in other embodiments, the different regions of extracellular microstructure are non-overlapping. This site-specific modulation is achieved by focusing a higher frequency ultrasound wave field to the desired sites of microstructure modulation. As described herein, applicants have demonstrated that a high frequency, narrow beam exposure geometry can be used to generate three-dimensional scaffolds having a central cylindrical core of short, thin, densely packed collagen fibers that is surrounded by periphery of long, thick, loosely packed collagen fibers. One of skill in the art readily appreciates that this approach can be employed to generate three-dimensional scaffolds having complex spatial patterning of various microstructures throughout the biological composition. For example, one or more vertical or horizontal columns throughout the composition can be formed using multiple ultrasound beams. Likewise, grid-like matrices can be formed by using an intersection of multiple ultrasound beams. As noted above, various transducer geometries (e.g., cylinders, tubes, ellipses, circles, rectangles, squares, triangles, spheres, rings, disks, plates/planes) can be utilized to achieve the desired spatial and patterned arrangement or organization of the ECM microstructures. When forming complex spatially patterned arrangements of ECM microstructure, it may be desirable to employ a mask to more precisely direct ultrasound exposure to defined areas of a composition. The mask would be fabricated from an acoustically-reflecting, or acoustically-absorbing, material such as, but not limited to, air-filled cork or metal. In this embodiment of the invention, the mask is place between the biological composition and the ultrasound source to selectively direct ultrasound exposure to defined regions of the biological composition.

The change in ECM protein microstructure observed upon ultrasound exposure enhances the mechanical strength of the biological compositions. For example, a biological composition exposed to ultrasound may have a 2-fold to 100-fold increase in mechanical strength compared to a biological composition that is not exposed to ultrasound. Increasing the mechanical strength of a tissue is especially desirable in the development of artificial tissues such as skin, cartilage, bone, arteries, veins, cardiac tissue, lungs, tendon, ligament, vascular tissues, and in tissues designed to regulate or control cell differentiation, e.g., stem cell differentiation.

In some embodiments of the present invention, it is desirable to produce a biological composition, e.g., a tissue scaffold or engineered tissue construct, having variations in mechanical strength. This can be achieved by site-specifically controlling the ECM protein microstructure throughout the biological composition as described supra. Consistent with this embodiment, resulting three-dimensional tissue scaffolds or tissue constructs having two or more regions that vary in mechanical strength can be generated using the methods of the present invention. Inter-tissue variations in mechanical strength are important for the fabrication of artificial striated tissues, e.g., tendons and ligaments, and layered tissues, e.g., articular cartilage.

Variations in ECM protein microstructure throughout a biological composition, and therefore, throughout the resulting three-dimensional tissue scaffold, result in variations in the cell growth substrate, which consequently modulates cell behavior. Cell behaviors that may be modified or influenced by the ECM microstructure of the tissue scaffold include, without limitation, cell survival, cell growth, cell differentiation, cell migration, and gene expression. For example, as demonstrated herein, ultrasound-induced changes in collagen fiber microstructures altered fibroblast adhesion and migration behavior. Accordingly, in one embodiment of the invention, ultrasound induced changes in ECM protein microstructure enhance or encourage cell specific adhesion and/or migration within and throughout spatially defined regions of the biological composition. The generation of three-dimensional tissue scaffolds having variation in the ECM microstructure facilitates the development of more complex tissue structures having complex cell spatial organizations, for example, liver or pancreas tissue. Additionally, tissue scaffolds can be designed to have site specific variations in ECM microstructure that promote cell specific behaviors within the scaffold, e.g., differentiation or migration. The ability to control cell specific migration in a site-specific manner is particularly useful in generating nerve tissue constructs, for example, facilitating the generation of constructs that can be used to direct nerve regeneration.

The methods of the present invention can be used to modify and enhance tissue engineering technologies previously developed in the art that utilize an extracellular matrix tissue scaffold or substrate (e.g., a hydrogel, matrigel, or collagen based substrate containing one or more unpolymerized ECM proteins). It is expected that application of the methods of the present invention to these systems will facilitate the generation of tissue scaffolds having defined ECM protein microstructures, which subsequently will generate more complex three dimensional tissues having expanded in vitro and, more importantly, in vivo utility. Examples of suitable three dimensional tissue engineered constructs to which the methods of the present invention are applicable to include, without limitation, oral tissue constructs (U.S. Patent Application Publication No. 20060171902 to Atala et al., which is hereby incorporated by reference in its entirety); cardiac constructs (U.S. Patent Application Publication No. 20080075750 to Akins and U.S. Pat. No. 5,885,829 to Mooney et al., which are hereby incorporated by reference in their entirety); embryonic brain tissue construct (U.S. Patent Application Publication No. 20060030043 to Ma, which is hereby incorporated by reference in its entirety); muscular construct (U.S. Patent Application Publication Nos. 20060198827 to Levenberg et al., 20060134076 to Bitar et al, and U.S. Pat. No. 6,537,567 to Niklason et al., which are hereby incorporated by reference in their entirety); stromal cell constructs (U.S. Pat. No. 4,963,489 to Naughton et al. and U.S. Patent Application Publication No. 20030007954 to Naughton et al., which are hereby incorporated by reference in their entirety); embryonic tissue constructs (U.S. Patent Application Publication No. 20050031598 to Levenberg et al., which is hereby incorporated by reference in its entirety); pancreatic constructs (U.S. Pat. No. 6,022,743 to Naughton et al., which is hereby incorporated by reference in its entirety); skin constructs (U.S. Pat. No. 5,266,480 to Naughton et al., which is hereby incorporated by reference in its entirety); filamentous tissue/ligament construct (U.S. Pat. No. 6,140,039 to Naughton et al., U.S. Pat. No. 6,840,962 to Vacanti et al., and U.S. Pat. No. 6,737,053 to Goh et al., which are hereby incorporated by reference in their entirety); cartilage constructs (U.S. Pat. No. 5,902,741 to Purchio et al., which is hereby incorporated by reference in its entirety); vascular constructs (U.S. Pat. No. 7,112,218 to McAllister et al., and U.S. Pat. No. 7,179,287 to Wolfinbarger, which are hereby incorporated by reference in their entirety); kidney constructs (U.S. Pat. No. 5,516,680 to Naughton et al., which is hereby incorporated by reference in its entirety); uterine constructs (U.S. Patent Application Publication No. 20030096406 to Atala et al., which is hereby incorporated by reference in its entirety); and liver constructs (U.S. Pat. No. 5,624,840 to Naughton et al., which is hereby incorporated by reference in its entirety).

Another aspect of the present invention is directed to a monolithic tissue scaffold comprising two or more spatially defined, integrated, non-overlapping regions, where at least two of the regions comprise different extracellular matrix protein microstructures. In accordance with this aspect of the invention, a monolithic tissue scaffold is a three-dimensional structure consisting of a single unit, i.e., it is formed from or composed of material without joints or seams (e.g., from a biological composition as described supra). The tissue scaffold of the invention can assume any three-dimensional shape, including, without limitation a cylinder, sphere, ellipsoid, disk, sheet, cube, cuboid, cone, triangular or rectangular prism, as well as hollow spheres, hollow ellipsoids, etc, and open-ended, hollow cylinders, etc.

Although monolithic in nature, the tissue scaffold of the present invention comprises one or more spatially defined integrated regions of variation with respect to extracellular matrix protein microstructure. In accordance with this aspect of the present invention, the integrated regions of the monolithic tissue scaffold are organized or structured so as to function cooperatively together as a whole tissue. In one embodiment of the invention, the tissue scaffold comprises a plurality of spatially patterned regions of variation. The spatially defined regions of variation can traverse the scaffold horizontally, vertically, or a combination thereof, and exist in any one of a variety of geometries, e.g., cylinders, tubes, grids, ellipses, circles, rectangles, squares, triangles, spheres, rings, disks, plates/planes. In a preferred embodiment, the monolithic tissue scaffold is made in accordance with the methods of the present invention. In other words, the tissue scaffold is made from a biological composition comprising one or more soluble unpolymerized extracellular matrix proteins. The method of generating the tissue scaffold involves adjusting the conditions of the biological composition to effect extracellular matrix protein polymerization, and exposing the biological composition to one or more ultrasound wave fields during extracellular matrix protein polymerization under conditions effective to control the extracellular matrix protein microstructure within the biological composition

As described above, variations in ECM protein microstructure cause variations in mechanical strength throughout the tissue scaffold. Accordingly, in one embodiment of this aspect of the present invention, the monolithic tissue scaffold comprises at least two regions having different mechanical strength. Likewise, variations in ECM protein microstructure differentially alter the cell substrate of the scaffold, which modifies cell behavior. Accordingly, in one embodiment of this aspect of the invention, the monolithic tissue scaffold comprises at least one region of ECM microstructure that permits cell adhesion. In another embodiment of the invention, the tissue scaffold comprises at least one region of ECM microstructure that encourages or induces cell migration. In another embodiment of the present invention, the tissue scaffold comprises at least one region of ECM microstructure that permits cell specific adhesion and a second region of ECM microstructure that encourages cell specific migration.

Another aspect of the present invention is directed to an engineered tissue construct comprising a monolithic tissue scaffold of the present invention, where the tissue scaffold has one or more regions of modified extracellular matrix microstructure. As used herein, an engineered tissue construct encompasses a three dimensional mass of living, artificially produced tissue, preferably mammalian tissue. The artificial tissue may be produced and maintained ex vivo; however, because it is designed to share critical structural and functional characteristics of intact tissue, it is suitable for in vivo implantation or transplantation into a suitable host in need of tissue replacement or repair. Incorporation of the tissue scaffold of the present invention, which more closely mimics the inhomogeneities naturally present within the extracellular matrix of native tissues, allows for the fabrication of more functionally complex engineered tissue constructs, i.e., constructs that better mimic intact tissue structure and function.

In accordance with this aspect of the invention, the engineered tissue construct comprises one or more cell types selected from the group consisting of smooth muscle cells, cardiac muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, and precursor cells. The tissue scaffold of the present invention, may have spatially defined regions of different extracellular matrix protein microstructure, allowing for the optimized growth of various different cell types within the artificial tissue construct. For example, in one embodiment of the invention a first cell type is localized to or within a first spatially defined region of the construct having a first extracellular matrix protein microstructure and a second cell type is localized to or within a second spatially defined region of the construct having a second extracellular matrix protein microstructure. Optionally, a third, fourth, fifth, etc, cell type can also be localized or co-localized to or within the first or second regions of the construct. Alternatively, these additional cell types can be localized to a third, fourth, or fifth spatially defined region of the construct. This sort of arrangement facilitates the engineering of tissues having complex cell spatial organizations, such as liver tissue or pancreatic tissue.

Cells can be cultured and maintained on or within the tissue scaffold of the present invention for the generation of an engineered tissue construct using standard tissue culture procedures. Appropriate growth and culture conditions for various mammalian cell types are well known in the art. The cells of the tissue construct may be seeded onto and/or within a substrate from a suspension so that they are evenly distributed at a relatively high surface and/or volume density. The cell suspensions may comprise approximately about 1×10⁴ to about 5×10⁷ cells/ml of culture medium, or approximately about 2×10⁶ cells/ml to about 2×10⁷ cells/ml, or approximately about 5×10⁶ cells/ml. The optimal concentration and absolute number of cells will vary with cell type, growth rate of the cells, substrate composition, including ECM protein microstructure, along with a variety of other parameters. The suspension may be formed in any physiologically acceptable medium, preferably one that does not damage the cells or impair their ability to adhere to the substrate. Appropriate mediums include standard cell growth media (e.g., DMEM with 10% FBS).

Examples of suitable seeding and culturing methods for the growth of three-dimensional cell cultures, including techniques for inoculating a three-dimensional matrix with the desired cells, and maintaining the culture are disclosed in U.S. Pat. No. 6,537,567 to Niklason et al., U.S. Pat. No. 5,266,480 to Naughton et al., and U.S. Pat. No. 5,770,417 to Vacanti et al., which are hereby incorporated by reference in their entirety.

In an alternative embodiment of the invention, cells of the engineered tissue construct of the present invention are suspended in the polymerizable biological composition and seeded into an appropriate culture dish or onto an appropriate substrate. In this embodiment, ultrasound exposure is used to both spatially organize the cells within the scaffold and modulate ECM protein microstructure. As a result, cell growth and other behaviors within the biological composition are optimized based on spatial organization within an environment that more closely mimics the in vivo tissue environment. This embodiment of the invention is particularly suitable for the construction of vascularized three-dimensional engineered tissues having spatially-defined variations in extracellular matrix protein microstructure. The fabrication of vascularized three-dimensional engineered tissue constructs using ultrasound exposure is described in WO/2010/135044 to Dalecki et al., which is hereby incorporated by reference in its entirety.

Cells of an engineered tissue construct are cultured in a media that generally includes essential nutrients and, optionally, additional elements such as growth factors, salts, minerals, vitamins, etc., that may be selected according to the cell type(s) being cultured. A standard growth media includes Dulbecco's Modified Eagle Medium, low glucose (DMEM), with 110 mg/L pyruvate and glutamine, supplemented with 10-20% fetal bovine serum (FBS) or calf serum and 100 U/ml penicillin. The culture media may also contain particular growth factors selected to enhance cell survival, differentiation, secretion of specific proteins, etc. In accordance with this aspect of the present invention, factors that enhance cell growth, proliferation, and differentiation may be added to culture medium or the biological composition comprising the tissue scaffold on which the cells are growing in or on.

The engineered tissue construct comprising the monolithic tissue scaffold of the present invention can be any desired tissue construct, including, but not limited to, a muscular tissue construct, a vascular tissue construct, an esophageal tissue construct, an intestinal tissue construct, a rectal tissue construct, an ureteral tissue construct, a cartilaginous tissue construct, a cardiac tissue construct, a liver tissue construct, a bladder tissue construct, a kidney tissue construct, a pancreatic tissue construct, a skeletal tissue construct, a filamentous/ligament tissue construct, a lung tissue construct, a neural tissue construct, a bone tissue construct, and a skin tissue construct.

In one embodiment of the present invention, the engineered tissue construct is a vascularized tissue construct having defined extracellular matrix protein microstructure. In accordance with this aspect of the present invention, the vascularized engineered tissue construct contains networks of microvessels that form an “exchange” network capable of supplying nutrients and removing wastes from the new tissue. Upon transplantation of the vascularized tissue construct of the present invention, the tissue construct integrates or connects to host tissue either by inducing incoming vessels into the construct (i.e., angioinduction) and/or by allowing the construct vessels to meet the host vessels (i.e., inosculation).

In another embodiment of the present invention, an engineered tissue construct is one having enhanced mechanical strength. An exemplary tissue construct where enhanced mechanical strength is desired is an artificial skin construct. In another embodiment of the present invention, an engineered tissue construct having variations in mechanical strength is produced. Exemplary constructs include striated tissues, such as tendons and ligaments or layered tissues, such a articular cartilage. The cell types that may be used to generate a three dimensional striated or layered tissue constructs include, without limitation, tenocytes, ligamentum cells, fibroblasts, chondrocytes, and endothelial cells. These cells may be used independently or in combination and may be primary cells or derived from cell lines.

Another embodiment of the present invention is directed to a method of treating a subject in need of tissue repair or tissue replacement. This method involves selecting a subject that is in need of tissue repair or tissue replacement and implanting a suitable engineered tissue construct of the present invention into the selected subject. In accordance with this aspect of the present invention, a suitable subject is any animal, preferably a mammal, more preferably, a human subject.

The engineered tissue constructs of the present invention may be used to replace or augment existing tissue, to introduce new or altered tissue, to modify artificial prostheses, or to join together biological tissues or structures. For implantation or transplantation in vivo, either the cells obtained from the engineered tissue construct or, more preferably, the entire three dimensional engineered tissue construct is implanted, depending on the type of tissue involved. In accordance with this aspect of the invention, it is desirable to use allogeneic cells, i.e., cells or tissue originated from or derived from a donor of the same species as the recipient, in the engineered tissue construct. It may further be desirable, when possible to use autologous cells, i.e., the cells and tissue are derived from the recipient.

The following examples illustrate various methods for compositions in the treatment method of the invention. The examples are intended to illustrate, but in no way limit, the scope of the invention.

### EXAMPLES

### Materials and Methods for Examples 1-7

Ultrasound Exposure Apparatus.

The experimental set-up used for ultrasound exposures (FIG. 1A) has been described previously (Garvin et al., “Controlling the Spatial Organization of Cells and Extracellular Proteins in Engineered Tissues Using Ultrasound Standing Wave Fields,” Ultrasound Med. Biol. 36(11):1919-32 (2010) and Garvin et al., “Vascularization of Three-Dimensional Collagen Hydrogels Using Ultrasound Standing Wave Fields,” Ultrasound Med. Biol. 37(11): 1853-64 (2011), which are hereby incorporated by reference in their entirety). Briefly, the acoustic source, either a 1-MHz (2.5 cm diameter) or 8.3-MHz (0.64 cm diameter) unfocused piezoceramic transducer, was mounted to the bottom of a plastic exposure tank filled with degassed, deionized water. The ultrasound signal driving the transducer was generated using a waveform generator (Hewlett Packard, Model 33120A, Palo Alto, Calif., USA or Agilent, Model 33250A, Santa Clara, Calif., USA), an attenuator (Kay Elemetrics, Model 837, Lincoln Park, N.J., USA), and a radio-frequency (RF) power amplifier (ENI, Model 2100L, Rochester, N.Y., USA). Samples were contained in the wells of silicone elastomer-bottomed cell culture plates (FlexCell International Corporation, FlexI® or BioFlex® plates, Hillsborough, N.C., USA). In some experiments, well diameters of FlexI® culture plates were reduced to 1 cm using elastomer molds (Sylgard® 184 silicone elastomer; Dow Corning Corporation, Midland, Mich., USA). Sample holders were mounted to a three-axis positioner (Velmex, Series B4000 Unislide, East Bloomfield, N.Y., USA) to allow precise control over sample location within the sound field. In this set-up, the presence of an air interface above the sample produced an ultrasound standing wave field within the sample volume (Garvin et al., “Controlling the Spatial Organization of Cells and Extracellular Proteins in Engineered Tissues Using Ultrasound Standing Wave Fields,” Ultrasound Med. Biol. 36(11):1919-32 (2010) and Garvin et al., “Vascularization of Three-Dimensional Collagen Hydrogels Using Ultrasound Standing Wave Fields,” Ultrasound Med. Biol. 37(11): 1853-64 (2011), which are hereby incorporated by reference in their entirety).

Acoustic field calibrations were conducted prior to each experiment using a needle hydrophone (Onda Corporation, HNC-0400, Sunnyvale, Calif., USA) under traveling wave conditions. Acoustic pressures were measured at axial distances of 12.2 cm from the transducer for experiments at 1 MHz, and 6.7 cm from the transducer for experiments at 8.3 MHz. The −6 dB transaxial beamwidths of the 1-MHz and 8.3-MHz sound fields at these axial locations were 1.2 cm and 0.6 cm, respectively. Sample holder placement within the acoustic field has been described previously (Garvin et al., “Controlling the Spatial Organization of Cells and Extracellular Proteins in Engineered Tissues Using Ultrasound Standing Wave Fields,” Ultrasound Med. Biol. 36(11):1919-32 (2010), which is hereby incorporated by reference in its entirety). Briefly, coordinates from a fixed point of reference to the field exposure location were obtained from the hydrophone calibration, and the three-axis positioner was used to locate well bottoms of the cell culture plates at the air-water interface, either 12.2 cm (1 MHz experiments) or 6.7 cm (8.3 MHz experiments) from the transducer. The ultrasound-exposed well of the sample holder was positioned so that the maximum acoustic pressure in the transaxial direction was centered within the well at the desired axial locations listed above.

In some experiments, the apparatus for ultrasound exposures was modified to produce a traveling wave field within the sample volume (FIG. 1B). The acoustic source (1 MHz, 2.5 cm diameter piezoceramic transducer) was mounted to the side of the plastic exposure tank. Samples were contained within plastic cuvettes (1 cm×1 cm×4.5 cm; VWR, Radnor, Pa., USA) mounted to the three-axis positioner. Cuvettes were modified by replacing the front and back walls with acoustically transparent silicone elastomer membranes (FlexCell). A rubber absorber was placed behind the sample holder to reduce reflections. The front face of the cuvette was situated at an axial distance of 12.2 cm from the transducer, and the maximum acoustic pressure in the transaxial direction at this location was centered within the lower 1 cm of the cuvette.

Collagen Sample Preparation.

Neutralized type I collagen solutions were prepared on ice by combining type I collagen (BD Biosciences, Bedford, Mass., USA; from rat tail) with 2× concentrated Dulbecco's modified Eagle's medium (DMEM; Invitrogen, Carlsbad, Calif., USA) and 1×DMEM such that the final mixture contained 1×DMEM and 0.8 mg/ml collagen (Garvin et al., “Controlling the Spatial Organization of Cells and Extracellular Proteins in Engineered Tissues Using Ultrasound Standing Wave Fields,” Ultrasound Med. Biol. 36(11):1919-32 (2010) and Garvin et al., “Vascularization of Three-Dimensional Collagen Hydrogels Using Ultrasound Standing Wave Fields,” Ultrasound Med. Biol. 37(11): 1853-64 (2011), which are hereby incorporated by reference in their entirety).

Ultrasound Exposures.

For ultrasound exposures using the apparatus shown in FIG. 1A, aliquots of the collagen solution were pipetted into 2 wells of the cell culture plate yielding samples 0.5 cm in height. One sample was exposed to ultrasound at either 1 MHz or 8.3 MHz for 15 min in a room temperature (RT) water tank. The other sample was treated exactly as the ultrasound-exposed sample but was not exposed to the sound field and served as the sham exposure condition. The 15-min exposure was sufficient for collagen polymerization. This set-up (FIG. 1A) produced an ultrasound standing wave field within the sample volume (Garvin et al., “Controlling the Spatial Organization of Cells and Extracellular Proteins in Engineered Tissues Using Ultrasound Standing Wave Fields,” Ultrasound Med. Biol. 36(11):1919-32 (2010) and Garvin et al., “Vascularization of Three-Dimensional Collagen Hydrogels Using Ultrasound Standing Wave Fields,” Ultrasound Med. Biol. 37(11): 1853-64 (2011), which are hereby incorporated by reference in their entirety). Acoustic pressures reported here are the maximum peak positive pressure at an antinode, and reported intensities are the spatial peak temporal average intensity (Iₛₚₜₐ) calculated at an antinode.

For experiments at 1 MHz, samples were subjected to either continuous wave (CW) or pulsed exposures (20-0 pulse duration, duty cycles of 0.12, 0.24, or 0.5) at various peak positive acoustic pressures (0-1 MPa) and spatial peak temporal average intensities (Iₛₚₜₐ=0-2.4 W/cm²). For experiments at 8.3 MHz, samples were exposed to CW ultrasound at Iₛₚₜₐ values of 0 or 30 W/cm².

Following ultrasound exposure, collagen gels were incubated at 37° C., 5% CO₂ for 20 min. Samples were then fixed for 1 hr using 4% paraformaldehyde and then washed 3× with PBS. In some experiments, collagen gels were fixed 20 hr after ultrasound exposure. In other experiments, the collagen solution was allowed to polymerize within sample holders at 37° C., 5% CO₂ for 1 hr prior to ultrasound exposure.

For traveling wave field exposures (FIG. 1B), an aliquot of the collagen solution was pipetted into the cuvette sample holder and exposed to, or not exposed to (sham condition) ultrasound (1 MHz) for 15 min in a room temperature water tank. Samples were exposed at 3 W/cm² Iₛₚₜₐ using either CW or pulsed (100 μs pulse duration, duty cycle of 0.5) ultrasound. Collagen gels were then fixed after a 20-min incubation at 37° C., 5% CO₂ as described above.

Microscopy.

To visualize type I collagen fibers, collagen gels were examined using either second-harmonic generation microscopy (Garvin et al., “Controlling the Spatial Organization of Cells and Extracellular Proteins in Engineered Tissues Using Ultrasound Standing Wave Fields,” Ultrasound Med. Biol. 36(11):1919-32 (2010), which is hereby incorporated by reference in its entirety) or scanning electron microscopy. Second-harmonic generation microscopy was performed using an Olympus Fluoview 1000 AOM-MPM microscope equipped with a 25×, 1.05 NA water immersion lens (Olympus, Center Valley, Pa., USA). Samples were illuminated with 780 nm light generated by a Mai Tai HP Deep See Ti:Sa laser (Spectra-Physics, Mountain View, Calif., USA) and the emitted light was detected with a photomultiplier tube using a bandpass filter with a 390 nm center wavelength (Semrock, Inc., Filter FF01-390/40-25, Rochester, N.Y., USA). Collagen fibers were photographed using a CMOS digital camera (Motic, Moticam 1000, Xiamen, China). For ultrasound standing wave field experiments at 1 MHz, images were collected in 5-μm steps in the z-direction through a 100-μm depth at both the first pressure antinode from the center of the gel surface (375 μm) and the first pressure node from the center of the gel surface (750 μm). Images were then projected onto the z-plane using ImageJ software (NIH, Bethesda, Md., USA) to create three-dimensional projections of collagen fiber morphology. For ultrasound standing wave field experiments at 8.3 MHz, additional sets of images were collected near the periphery of the collagen gel. For traveling wave field experiments, images were collected at a depth of 900-1000 μm from the center of the front face of the gel surface.

Scanning electron microscopy was performed using a Zeiss Auriga focused ion beam-scanning electron microscope equipped with high vacuum secondary electron imaging and TIFF file recording (Carl Zeiss Microscopy, Peabody, Mass., USA). Collagen samples were prepared for electron microscopy by fixation in 2% glutaraldehyde followed by dehydration in a graded series of ethanol washes. Samples were washed in a graded series of hexamethyldisilazane to sequentially exchange the ethanol and were allowed to air dry. Dried collagen samples were mounted on scanning electron microscope sample stages and sputter coated with gold to an approximate thickness of 3 nm (Desk II Sputter Coater, Denton Vacuum, LLC, Moorestown, N.J., USA). Prepared collagen samples were imaged at 10 kV and magnifications of 10,000× and 50,000×.

Quantification of Collagen Fiber Density and Diameter.

Images collected using second-harmonic generation microscopy were analyzed using ImageJ software (NIH) to quantify density of type I collagen fiber networks. For each z-stack of images, images were thresholded and converted from grayscale to binary using an Otsu black and white filter. The number of white pixels in each image series, representing collagen fibers, was divided by the total number of black and white pixels to calculate collagen fiber density. A total of three z-stacks of images were analyzed at both imaging locations (pressure antinode and node) within three separate collagen gels per condition that were fabricated on three different experimental days.

Scanning electron microscopy images were analyzed using ImageJ software to quantify collagen fiber diameter. A 64×64 pixel grid was overlaid onto images collected at 50,000× magnification. At each grid intersection point, collagen fiber diameter was measured. A total of 18 images per condition were used to quantify collagen fiber diameters. Three images were collected from each of 6 samples per condition that were fabricated on three different experimental days. For each collagen sample, a histogram of fiber diameters was compiled to display the frequency of occurrence of fiber diameters as a percentage of the total number of fibers measured.

Thermocouple Measurements.

Temperature changes within collagen samples were monitored during ultrasound exposure using a 50-μm copper-constantan thermocouple situated at the location of maximum acoustic pressure within the samples. Temperature was monitored using a digital laboratory thermometer (Physitemp Instruments Inc., Model BAT-12, Clifton, N.J., USA), sensitive to changes of 0.1° C. For exposures at 8.3 MHz, temperature changes at both the gel center (maximum acoustic pressure) and gel periphery were measured simultaneously using two thermocouples and thermometers.

Cell Culture and Cell Seeding onto Ultrasound-Exposed Collagen Gels.

Fibronectin-null mouse embryonic myofibroblasts (MEFs) were cultured in a 1:1 mixture of AimV (Invitrogen) and CellGro (Mediatech, Herndon, Va., USA) on tissue culture dishes pre-coated with type I collagen, as described previously (Garvin et al., “Controlling the Spatial Organization of Cells and Extracellular Proteins in Engineered Tissues Using Ultrasound Standing Wave Fields,” Ultrasound Med. Biol. 36(11):1919-32 (2010), which is hereby incorporated by reference in its entirety). Fibronectin-null MEFs were seeded at a density of 5.2×10⁴ cells/cm² onto ultrasound- and sham-exposed collagen gels and cultured at 37° C. and 8% CO₂. Cells were observed at various times using an Olympus BX60 microscope and images were collected using a digital camera (QImaging, ExiBlue, Surrey, BC, Canada).

Collagen fiber structure of cell-seeded collagen samples was examined using second-harmonic generation microscopy. Fibronectin-null MEFs were simultaneously visualized using a second bandpass filter with a 519 nm center wavelength (Olympus, Filter BA 495-540HQ from MPFC1) by exploiting the intrinsic auto-fluorescence of cells. Images were collected in the z-direction in 1-μm steps from the gel surface to a depth of 150 μm into the collagen sample.

Statistics.

Unless otherwise noted, all experiments were performed on three separate occasions. Data are presented as mean±SEM. Statistical comparisons between experimental conditions were performed using one-way analysis of variance in GraphPad Prism software (LaJolla, Calif., USA). Differences were considered significant for p values<0.05.

### Example 1

### Ultrasound Exposure Effects Collagen Fiber Structure

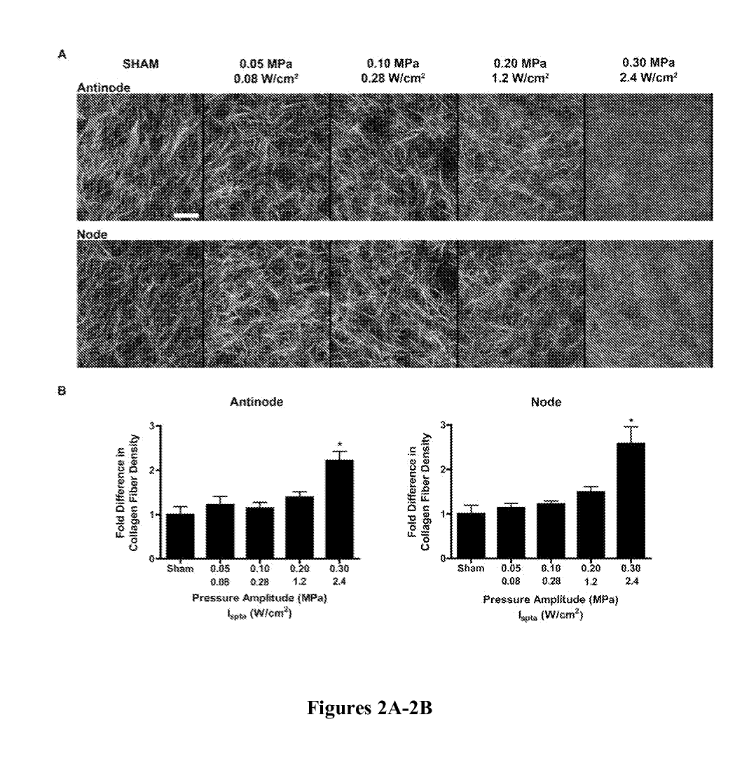

To investigate effects of ultrasound on collagen fiber microstructure, unpolymerized solutions of type I collagen were exposed to 1 MHz, CW ultrasound of various peak positive pressures and intensities using the apparatus diagramed in FIG. 1A. This exposure geometry produces an acoustic standing wave field throughout the collagen sample volume (Garvin et al., “Controlling the Spatial Organization of Cells and Extracellular Proteins in Engineered Tissues Using Ultrasound Standing Wave Fields,” Ultrasound Med. Biol. 36(11):1919-32 (2010), which is hereby incorporated by reference in its entirety). Therefore, collagen fiber morphology of polymerized gels was assessed at imaging depths corresponding to either pressure antinodes or nodes using second-harmonic generation microscopy imaging. Sham-exposed collagen gels were characterized by long, thick, loosely packed collagen fibers (FIG. 2A). In contrast, as the ultrasound exposure pressure increased, collagen fibers became visibly thinner, shorter, and more numerous at both pressure antinodes (FIG. 2A, upper panel) and nodes (FIG. 2A, lower panel). The onset for visible differences in collagen fiber structure occurred at 0.2 MPa, as measured at a pressure antinode (FIG. 2A), which corresponds to a spatial peak, temporal average intensity (Iₛₚₜₐ) of 1.2 W/cm².

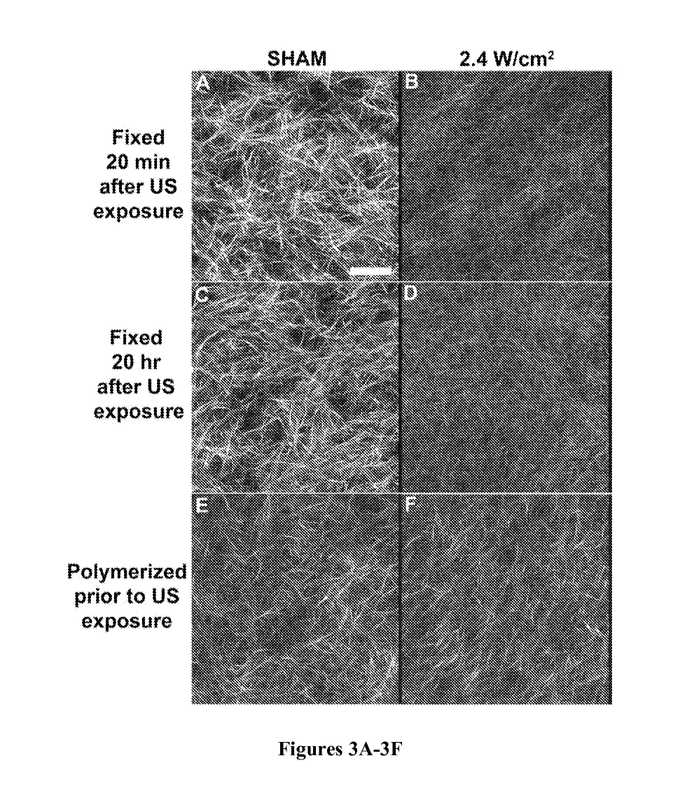

Changes in collagen fiber morphology were quantified through image processing (FIG. 2B). Results indicated a ˜1.5-fold, and ˜2.4-fold increase in fiber density over sham for samples exposed at 0.2 and 0.3 MPa, respectively. Similar changes in collagen fiber structure were observed at depths corresponding to both antinodes and nodes (FIG. 2B). Ultrasound-induced changes in collagen fiber microstructure persisted for at least 20 hr after exposure (FIG. 3), indicating that the observed changes were not readily reversible. Collagen gels polymerized prior to ultrasound exposure did not show changes in collagen microstructure compared to sham samples (FIG. 3).

### Example 2

### Ultrasound-Mediated Changes in Collagen Fiber Structure Depend on Iₛₚₜₐ

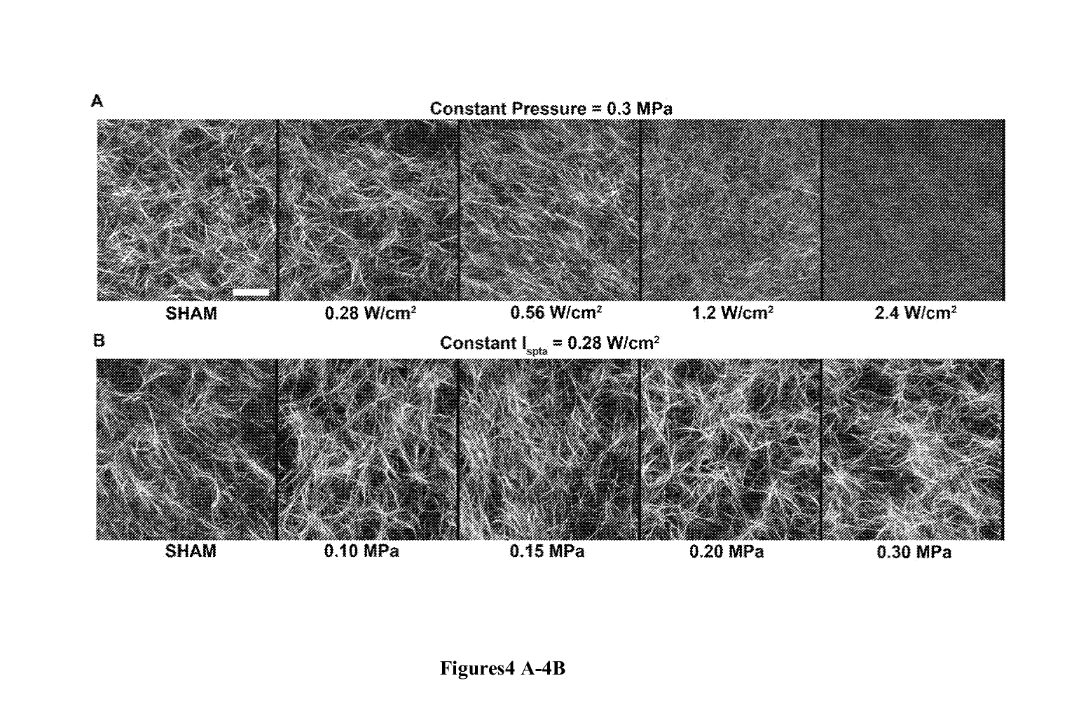

Other studies have shown that collagen fiber microstructure is affected by polymerization temperature (Roeder et al., “Tensile Mechanical Properties of Three-Dimensional Type I Collagen Extracellular Matrices With Varied Microstructure,” J. Biomech. Eng. 124:214-222 (2002); Sander & Barocas, “Biomimetic Collagen Tissues: Collagenous Tissue Engineering and Other Applications” in P. Fratzl, ed., Collagen Structure and Mechanics, Springer, New York, N.Y., pp. 475-504 (2008), which are hereby incorporated by reference in their entirety). Thus, experiments were conducted to investigate the role of ultrasound-induced heating in the observed effects on collagen fiber microstructure. Two investigations were performed to independently assess the roles of acoustic pressure and Iₛₚₜₐ. To examine the role of Iₛₚₜₐ, solutions of type I collagen were exposed to 1-MHz ultrasound standing wave fields of various Iₛₚₜₐ but constant pressure amplitude. A constant peak positive pressure amplitude of 0.3 MPa was utilized as this value was above the threshold for ultrasound-mediated changes in collagen fiber structure (FIG. 2). The ultrasound was pulsed using a 20-μs pulse duration and duty cycles of 0 (sham), 0.12, 0.24, 0.5, or 1 (CW), corresponding to Iₛₚₜₐ values of 0, 0.28, 0.56, 1.2, and 2.4 W/cm², respectively. Collagen gels exposed to ultrasound at or above 1.2 W/cm² consisted of thinner, shorter, and more densely packed fibers compared to sham-exposed gels (FIG. 4A). Similar results were observed at imaging depths corresponding to pressure antinodes (shown in FIG. 4A) and nodes.

To next investigate the role of acoustic pressure, solutions of type I collagen were exposed to an ultrasound standing wave field at 1-MHz with various pressure amplitudes but at a constant Iₛₚₜₐ (0.28 W/cm²), which was below the threshold for ultrasound-induced effects (FIG. 4A). The exposures were pulsed using a 20-μs pulse duration and duty cycles of 0 (sham), 0.12, 0.24, 0.5, or 1 (CW), corresponding to acoustic pressure amplitudes of 0, 0.3, 0.2, 0.15, and 0.1 MPa, respectively. No differences in collagen fiber structure were observed for any of the ultrasound-exposed gels compared to sham-exposed collagen samples (FIG. 4B), including those exposed at a pressure amplitude of 0.3 MPa. Taken together, these data indicate that Iₛₚₜₐ provides a predictor of ultrasound-induced changes in collagen fiber structure, suggesting that the effects of ultrasound on collagen microstructure are mediated in part through a thermal mechanism.

### Example 3

### Temperature Measurements and Thermal Effects of Ultrasound

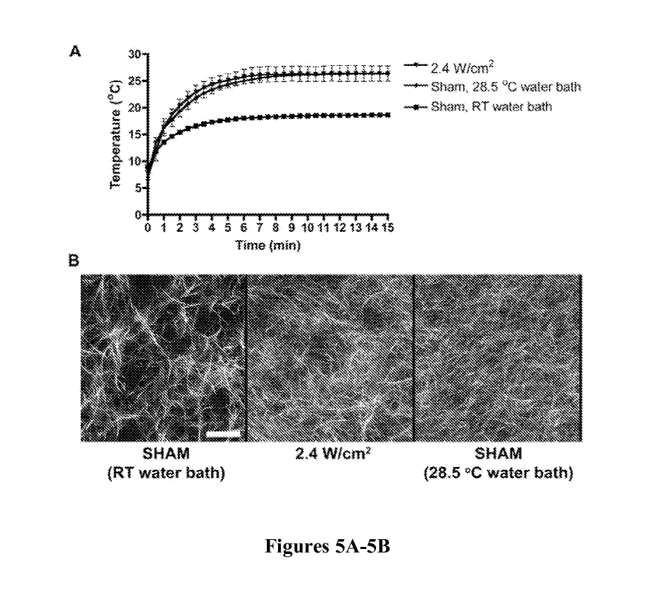

Temperatures of collagen gels were measured during ultrasound exposure. Sham-exposed gels reached a steady-state temperature of 18.6±0.3° C. (FIG. 5A; ‘Sham, RT water bath’). In contrast, the steady-state temperature within collagen gels exposed at 2.4 W/cm² was 26.3±1.7° C. (FIG. 5A). This temperature rise was simulated in sham-exposed samples by bulk heating using a water bath heated to 28.5° C. (FIG. 5A). The morphology of collagen fibrils of gels polymerized in the 28.5° C. water bath was similar to that of collagen gels exposed to ultrasound at 2.4 W/cm² (FIG. 5B). In these gels, fibrils appeared thinner, shorter and more densely packed than those of sham-exposed collagen gels polymerized at room temperature (FIG. 5B). Quantitative analysis of images showed a 2.5-fold increase in fiber density for samples polymerized in the 28.5° C. water tank and ultrasound-exposed samples at 2.4 W/cm², as compared to sham collagen gels polymerized at room temperature.

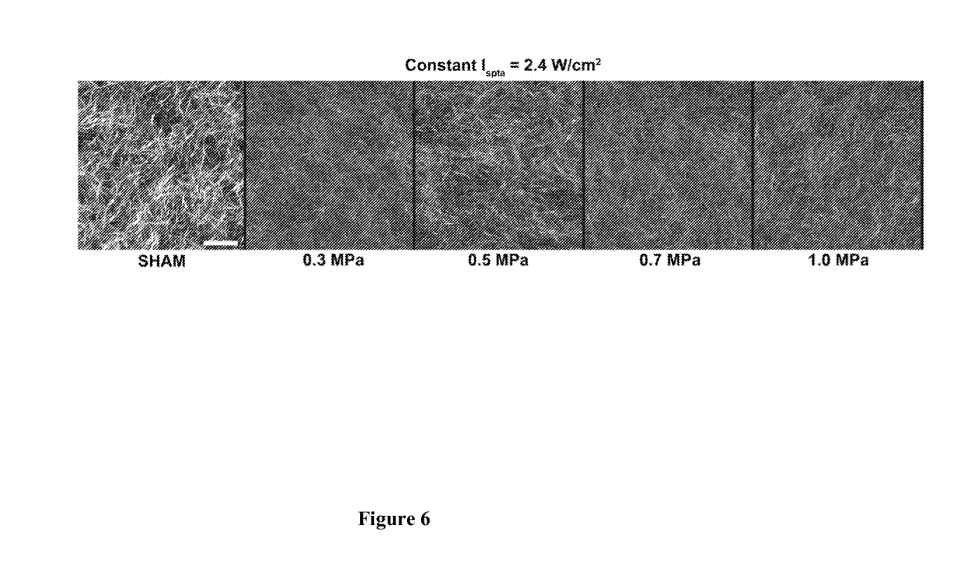

As a further test of the role of ultrasound-induced heating in collagen fiber morphology, solutions of type I collagen were exposed during polymerization to 1 MHz ultrasound standing wave fields of varying pressure but constant Iₛₚₜₐ (2.4 W/cm²), using pulsed exposures with a 20-μs pulse duration and duty cycles of 0 (sham), 0.12, 0.24, 0.5, or 1 (CW). Duty cycles of 0, 0.12, 0.24, 0.5, and 1 corresponded to acoustic pressures of 0, 1.0, 0.7, 0.5, and 0.3 MPa, respectively. Each of the ultrasound exposures produced nearly identical temperature profiles with final temperatures of ˜28° C.; this was expected given that the Iₛₚₜₐ was the same for each condition. Similar dense, short collagen fibrils were observed in all ultrasound-exposed samples compared to the thicker fibers produced in sham gels polymerized at 19° C. in a room temperature water bath (FIG. 6). These studies suggest that ultrasound-induced heating during collagen polymerization plays a roll in collagen fiber microstructure within hydrogels.

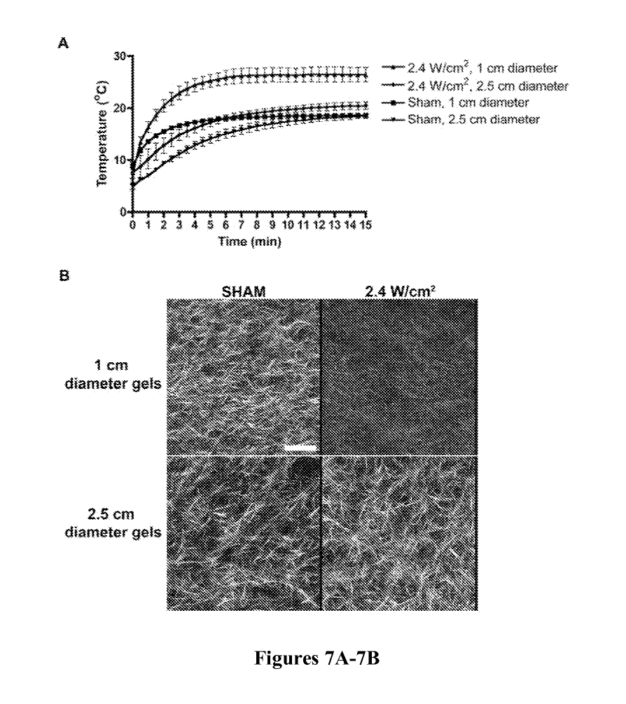

The magnitude of the heating observed in the measurements above was not a result of direct absorption of ultrasound in the collagen. The −6 dB beam width (1.2 cm) of the 1-MHz ultrasound field at the exposure site was comparable to the diameter (1 cm) of the Sylgard® mold used to contain the collagen gel. Sylgard® is a polymer with an absorption coefficient of 1.4±0.03 dB/cm at 1 MHz (Garvin et al., “Controlling the Spatial Organization of Cells and Extracellular Proteins in Engineered Tissues Using Ultrasound Standing Wave Fields,” Ultrasound Med. Biol. 36(11):1919-32 (2010), which is hereby incorporated by reference in its entirety). Thus, it was hypothesized that ultrasound-induced heating of the Sylgard® mold was the predominant source of thermal increases within the collagen samples in the above experiments. To test this, the Sylgard® mold was removed from the sample holder wells, and collagen gel samples were exposed to ultrasound in wells that were 2.5 cm in diameter. In these samples, the collagen solution filled the 2.5 cm diameter well and thus, only ultrasound-induced heating of the collagen, if any, was measured. Temperature profiles of collagen gels exposed to ultrasound at 2.4 W/cm² in either 1-cm or 2.5-cm wells are shown in FIG. 7A. Sham and ultrasound-exposed 2.5-cm diameter gels showed nearly identical increases in temperature to ˜19° C. (FIG. 7A), indicating limited ultrasound heating of collagen gels under these exposure conditions. No differences in collagen fiber structure were observed in gels exposed to ultrasound in the 2.5 cm wells compared to sham conditions (FIG. 7B). This lack of an effect on collagen fiber microstructure is consistent with the lack of ultrasound-induced temperature rise in the collagen samples contained within the larger wells under the stated exposure condition.

### Example 4

### Ultrasound Traveling Wave Field Exposures

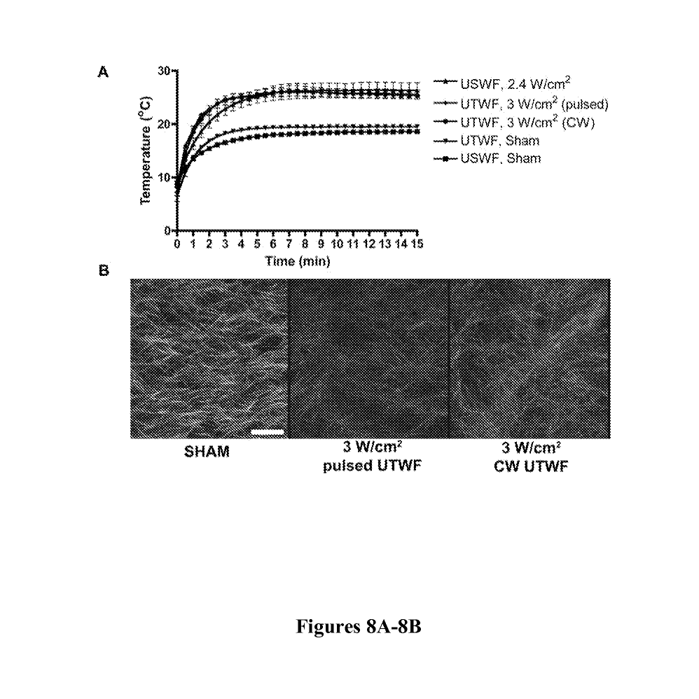

In the studies described thus far, similar changes in collagen fiber morphology were observed at both the pressure antinodes and nodes of the sample, suggesting that the ultrasound-induced effects are not dependent upon the standing wave field. To clearly demonstrate that standing wave fields are not necessary for effects of ultrasound on collagen fiber structure, the exposure geometry was reconfigured such that the collagen samples were exposed to an ultrasound traveling wave field (FIG. 1B). Temperature profiles of collagen gel samples exposed to ultrasound traveling wave fields or standing wave fields and the corresponding sham conditions are shown in FIG. 8A. Temperature profiles were similar for collagen solutions exposed to ultrasound using the following three conditions: i) 1 MHz, CW ultrasound standing wave field with Iₛₚₜₐ of 2.4 W/cm² at a pressure antinode; ii) 1 MHz, CW ultrasound traveling wave field with Iₛₚₜₐ of 3 W/cm²; and iii) 1 MHz, pulsed (100-μs pulse duration, duty cycle of 0.5) ultrasound traveling wave field with Iₛₚₜₐ of 3 W/cm² (FIG. 8A). For all ultrasound exposure conditions, the final steady-state temperature of the gels was approximately 25° C. (FIG. 8A). Collagen fibers of samples exposed to both ultrasound traveling wave fields conditions (pulsed and CW) were short, thin, and dense as compared to sham conditions (FIG. 8B), and comparable to collagen samples exposed to ultrasound standing wave fields at 2.4 W/cm² (FIG. 5B). These results demonstrate that effects of ultrasound on collagen fiber structure are not unique to either standing wave fields or traveling wave fields. Rather, it is the extent of the resultant ultrasound-induced heating during polymerization that noninvasively controls collagen fiber structure.

### Example 5

### Spatial Patterning of Collagen Microstructure Using Ultrasound

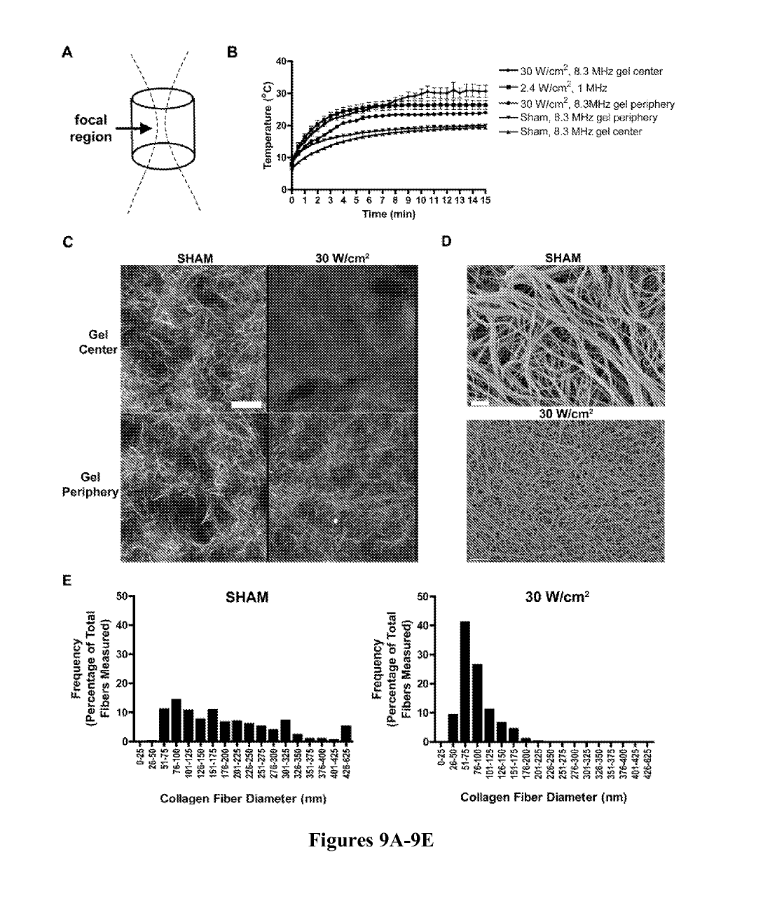

The absorption coefficient of ultrasound in soft-tissues is approximately proportional to acoustic frequency. Thus, to heat collagen directly with ultrasound, a higher frequency ultrasound source was employed. For these studies, an 8.3-MHz source with a −6 dB beam width of 0.6 cm at the exposure location was used. FIG. 9A illustrates the dimensions of the ultrasound beam width that was located centrally in a 4-cm diameter well containing the collagen solution. It was hypothesized that collagen microstructure could be controlled site-specifically such that ultrasound-induced heating within the central region of the gel would produce short, dense collagen fibers while longer, thicker fibrils would be produced outside the beam width, at the gel periphery. Temperature measurements were obtained at the center and periphery of collagen samples contained within 4-cm wells and exposed at 8.3 MHz, CW, and Iₛₚₜₐ of 30 W/cm² (FIG. 9B). As expected, temperatures in the center of samples exposed to ultrasound were significantly higher than temperatures at the periphery (FIG. 9B). Furthermore, temperatures in the center of these gels were comparable to temperatures measured within gels exposed at 1 MHz and 2.4 W/cm² and contained within 1-cm diameter Sylgard® molded wells (FIG. 9B).

Second-harmonic generation images were obtained at the center and periphery of polymerized collagen samples exposed to 8.3-MHz ultrasound within the larger wells. As expected from temperature profiles, dense networks of short, thin collagen fibers were observed within the central core of the collagen gel (FIG. 9C). In contrast, longer, thicker fibers were found outside the central ultrasound beam area (FIG. 9C). These experiments demonstrate the ability to design ultrasound exposure conditions to control collagen fiber structure noninvasively and site-specifically within a three-dimensional hydrogel.

Scanning electron microscopy images revealed decreased collagen fibril diameter in response to ultrasound exposure compared to sham (FIG. 9D), confirming changes observed by second-harmonic generation microscopy. Quantification of collagen fiber diameter from scanning electron microscopy images revealed that sham-exposed collagen samples contained a uniform distribution of fiber diameters ranging from ˜25 nm-600 nm (FIG. 9E, left panel). In contrast, ultrasound exposure shifted the collagen fiber diameter distribution to a higher occurrence of thinner fibers with sizes ranging from ˜25 nm-200 nm (FIG. 9E, right panel). Importantly, the absence of thick (200 nm-600 nm) fibrils in ultrasound-exposed collagen samples provides additional evidence that ultrasound exposure produces thinner collagen fibers within these collagen hydrogels.

### Example 6

### Cellular Response to Ultrasound-Exposed Collagen Hydrogels

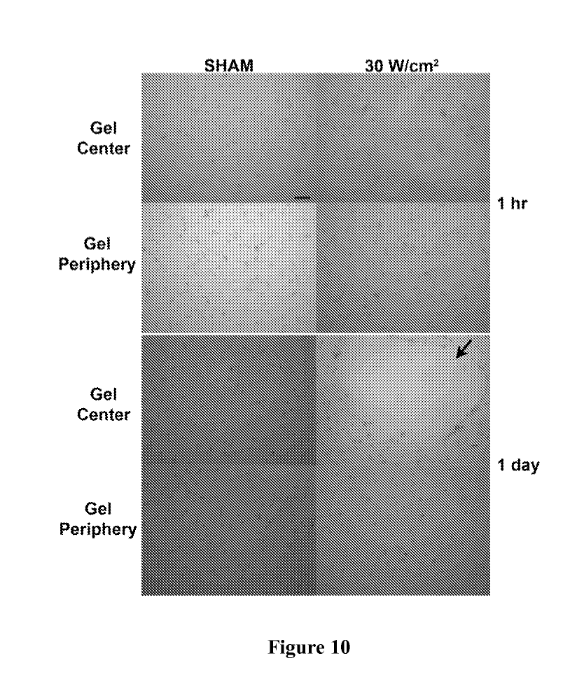

Collagen fiber structure affects cell behaviors that are critical to the fabrication of functional engineered tissue in vitro (Gelse et al., “Collagens—Structure, Function, and Biosynthesis,” Adv. Drug Deliv. Rev. 55:1531-1546 (2003), which is hereby incorporated by reference in its entirety). As such, studies were conducted to determine whether ultrasound-induced changes in collagen fiber microstructure would produce spatially-defined differences in cell behavior. For these experiments, fibronectin-null embryonic myofibroblasts (MEFs) were seeded onto 4-cm diameter collagen gels that had been exposed to ultrasound at 8.3 MHz and 30 W/cm². As shown in FIG. 9, this exposure produces a central cylinder (˜1 cm diameter) of dense, thin, short collagen fibrils within the 4-cm diameter gel. Fibronectin-null MEFs were utilized as they do not produce fibronectin and are cultured under serum-free conditions. Thus, initial cell adhesion in these experiments is mediated solely by the type I collagen substrate. Fibronectin-null MEFs adhered equally well to sham- and ultrasound-exposed collagen gels at both the gel center and periphery (FIG. 10, 1 hr). Immediately after seeding, cells were homogeneously distributed over both the central and peripheral regions of ultrasound-exposed gels (FIG. 10, 1 hr). However, within 1 day of seeding, cells within the central, ultrasound-exposed region of the collagen gel had migrated into small, circularly arranged aggregates (FIG. 10, 1 day, arrows). This spatial pattern remained evident up to 28 days after seeding. In contrast, cells seeded onto sham-exposed collagen, or cells adherent to the periphery of ultrasound-exposed samples, remained in a homogeneous distribution of single cells (FIG. 10, 1 day).

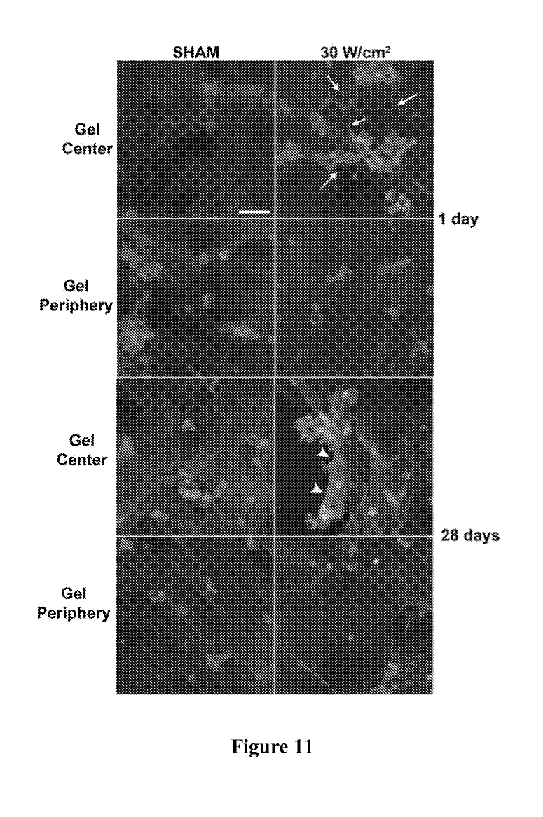

To examine the morphology of collagen fibers of cell-seeded collagen gels, second-harmonic generation microscopy images were collected at various times post-cell seeding. Cellular aggregates were present at the center of ultrasound-exposed samples at 1 day, and cells within this region reorganized their underlying collagen substrate into collagen bundles aligned between neighboring cell clusters (FIG. 11, 1 day, arrows). Over a 28-day period, cells extensively remodeled only the central, ultrasound-exposed collagen fibrils into dense sheet-like structures (FIG. 11, 28 days, arrowheads). This cell-mediated collagen reorganization was absent at the periphery of ultrasound-exposed gels (FIG. 11, 28 days). Similarly, collagen fibril structure of sham-exposed, cell-seeded gels was similar 1 and 28 days post-cell seeding (FIG. 11), indicating little cell-mediated reorganization of the collagen fibrils in sham samples. These data indicate that cells specifically remodel ultrasound-exposed collagen fibrils into dense collagen sheets. Furthermore, these results provide evidence that cells are capable of sensing ultrasound-induced changes in collagen microstructure, and can respond to localized variations in fiber structure within the same three-dimensional hydrogel by exhibiting spatial differences in cell behavior

### Example 7

### Thermal Independent Effects on Collagen Microstructure Induced by Ultrasound Exposure

As described in Example 3 above, the collagen microstructure in gels exposed to ultrasound during polymerization appears similar to the collagen microstructure in gels that are heated during the polymerization process. However, to investigate whether the resulting collagen microstructures were functionally the same, mouse embryonic myofibroblast migration on gels polymerized at 37° C. in the absence of ultrasound exposure was compared to cell migration on gels exposed to ultrasound during polymerization.

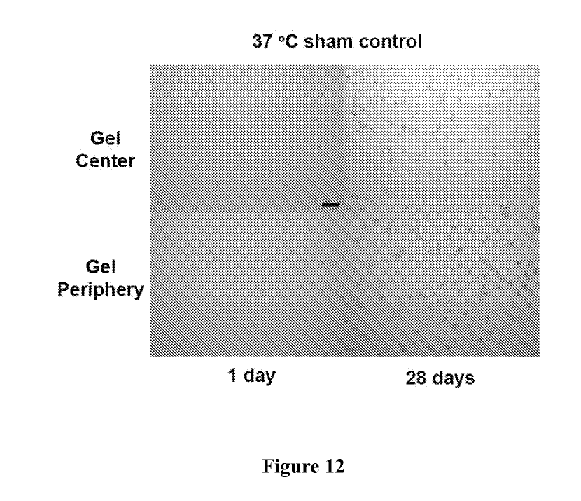

FIG. 12 provides evidence that the resulting collagen microstructures are not functionally similar. The phase contrast images of FIG. 12 are of mouse embryonic myofibroblasts seeded and grown on collagen gels fabricated at 37° C. in the absence of ultrasound exposure for 1 and 28 days. These representative images show that cells remain rounded and homogenously dispersed over the collagen substrate and do not migrate into small, circularly arranged aggregates as seen in response to 8.3-MHz ultrasound exposure (see FIG. 10 cells grown at gel center at 1 day). Accordingly, thermal mechanisms alone are not solely responsible for the cellular response to collagen gels fabricated using ultrasound. These data suggest that the collagen microstructure generated from ultrasound exposure is structurally distinct from the collagen microstructure generated by exposure to heat alone.

### Discussion of Examples 1-7

Examples 1-7 demonstrate that ultrasound can be used to noninvasively control the microstructure of collagen fibers within three-dimensional hydrogels. Under appropriate conditions, exposure of soluble collagen to ultrasound during the self-assembly process resulted in collagen gels with shorter and thinner fibers compared to collagen gels that were not exposed to ultrasound. These changes in collagen microstructure were produced using both ultrasound standing wave fields and traveling wave fields. The observed effects were localized to the ultrasound beam area and occurred throughout the three-dimensional gel volume. The effect of ultrasound on collagen microstructure occurred only when soluble collagen was exposed during the polymerization process; no effects of ultrasound on collagen fiber microstructure were observed in collagen gels that were polymerized prior to ultrasound exposure. The ultrasound-induced alterations in collagen microstructure were not transient or readily reversible. Braaten et al. “Ultrasound Reversibly Disaggregates Fibrin Fibers,” Thromb. Haemost. 78:1063-1068 (1997), which is hereby incorporated by reference in its entirety, observed changes in the microstructure of fibrin in fibrin clots exposed to ultrasound. In that work, effects of ultrasound on fibrin structure were transient.

Results of a series of mechanistic experiments were consistent with a thermal mechanism for the effects of ultrasound on collagen fiber microstructure. Ultrasound exposure conditions utilized in some experiments produced indirect heating of collagen samples due to absorption of sound in the elastomer mold surrounding the collagen. In other experiments, the mold was removed and a higher frequency and intensity were employed to heat the collagen directly. In all cases, the ultrasound-induced heating was relatively mild, producing final temperatures within the collagen gel of ˜30° C. for the highest intensities investigated in this study.

These results are consistent with literature reports demonstrating that collagen polymerized at different temperatures results in different fiber structures (Wood, G. C., “The Formation of Fibrils From Collagen Solutions. 2. A Mechanism of Collagen-Fibril Formation,” Biochem. J. 75:598-605 (1960), which is hereby incorporated by reference in its entirety). During the self-assembly process, collagen fiber thickness is affected by both pH and temperature, where lower pH and temperature provides a longer nucleation phase to produce thicker fibrils (Wood, G. C., “The Formation of Fibrils From Collagen Solutions. 2. A Mechanism of Collagen-Fibril Formation,” Biochem. J. 75:598-605 (1960); McPherson et al., “Collagen Fibrillogenesis In Vitro: A Characterization of Fibril Quality As a Function of Assembly Conditions,” Coll. Relat. Res. 5:119-135 (1985), which are hereby incorporated by reference in their entirety). Interestingly, the mechanical stiffness of collagen hydrogels is dependent upon collagen fiber microstructure, where thinner fibers result in stiffer gels (Roeder et al., “Tensile Mechanical Properties of Three-Dimensional Type I Collagen Extracellular Matrices With Varied Microstructure,” J. Biomech. Eng. 124:214-222 (2002); Raub et al., “Noninvasive Assessment of Collagen Gel Microstructure and Mechanics Using Multiphoton Microscopy,” Biophys. J. 92:2212-2222 (2007); Yang et al., “Elastic Moduli of Collagen Gels Can Be Predicted From Two-Dimensional Confocal Microscopy,” Biophys. J. 97:2051-2060 (2009), which are hereby incorporated by reference in their entirety). Physical parameters of collagen gels, such as fiber density, and bulk gel stiffness affect cell proliferation, viability, differentiation, and migration (Hansen et al., “Regulation of Hepatocyte Cell Cycle Progression and Differentiation by Type I Collagen Structure,” Curr. Top. Dev. Biol. 72:205-236 (2006); Sung et al., “Control of 3-Dimensional Collagen Matrix Polymerization for Reproducible Human Mammary Fibroblast Cell Culture in Microfluidic Devices,” Biomaterials 30:4833-4841 (2009); Baker et al., “The Influence of an Aligned Nanofibrous Topography on Human Mesenchymal Stem Cell Fibrochondrogenesis,” Biomaterials 31:6190-6200 (2010); Miron-Mendoza et al., “The Differential Regulation of Cell Motile Activity Through Matrix Stiffness and Porosity in Three Dimensional Collagen Matrices,” Biomaterials 31:6425-6435 (2010), which are hereby incorporated by reference in their entirety). Thus, there has been strong interest in developing technologies that can tune the physical properties of collagen gels to control cell behavior (Cen et al., “Collagen Tissue Engineering: Development of Novel Biomaterials and Applications,” Pediatr. Res. 63:492-496 (2008), which is hereby incorporated by reference in its entirety).

Ultrasound holds numerous technological advantages over bulk heating. The magnitude of heating produced by ultrasound can be controlled noninvasively through design of acoustic exposure parameters. Additionally, ultrasound heating can be produced site-specifically resulting in a single collagen hydrogel with spatial variations in fiber microstructure. Further, more complex spatial patterns of collagen microstructure within a hydrogel could be produced with the use of multiple focused ultrasound fields. In the present study, a high frequency ultrasound beam was directed within the center of a large collagen sample producing dense networks of short, thin collagen fibrils within the central core of the gel, and longer, thicker fibers outside the beam area. Fibroblasts seeded onto these gels migrated rapidly into small, circularly arranged aggregates only within the beam area, and clustered fibroblasts remodeled the central, ultrasound-exposed collagen fibrils into dense sheets. The observed differences in cell motility and collagen fibril remodeling activity likely occurred in response to regional differences in collagen gel stiffness (Zaman et al., “Migration of Tumor Cells in 3D Matrices is Governed by Matrix Stiffness Along With Cell-Matrix Adhesion and Proteolysis,” Proc. Nat'l. Acad. Sci. U.S.A. 103:10889-10894 (2006); Hadjipanayi et al., “Guiding Cell Migration in 3D: A Collagen Matrix With Graded Directional Stiffness,” Cell Motil. Cytoskeleton 66:121-128 (2009), which are hereby incorporated by reference in their entirety) and/or collagen fiber density (Grinnell & Petroll, “Cell Motility and Mechanics in Three-Dimensional Collagen Matrices,” Annu. Rev. Cell Dev. Biol. 26:335-361 (2009), which is hereby incorporated by reference in its entirety). Thus, ultrasound technologies that can noninvasively and site-specifically control the microstructure of collagen fibrils have the potential to produce three-dimensional, scaffolds with defined mechanical and biological properties.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1.-24. (canceled)

25. A monolithic tissue scaffold comprising two or more spatially defined, non-overlapping regions, wherein at least two of the regions comprise different extracellular matrix protein microstructures.

26. The monolithic tissue scaffold of claim 25, wherein the spatially defined, non-overlapping regions comprise one or more geometries selected from the group consisting of cylindrical, tubes, grids, ellipses, circles, rectangles, squares, triangles, spheres, rings, disks, plates/planes.

27. The monolithic tissue scaffold of claim 25, wherein the non-overlapping regions of the tissue scaffold having different extracellular matrix protein microstructures vary in mechanical strength.

28. The monolithic tissue scaffold of claim 25, wherein at least one of the non-overlapping regions of extracellular protein microstructure permits cell adhesion.

29. The monolithic tissue scaffold of claim 25, wherein at least one of the spatially defined, non-overlapping regions of extracellular protein microstructure permits cell migration.

30. An engineered tissue construct comprising the monolithic tissue scaffold of claim 25.

31. The engineered tissue construct of claim 30 comprising one or more cell types selected from the group consisting of smooth muscle cells, cardiac muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, and precursor cells.

32. The engineered tissue construct of claim 31, wherein the construct comprises at least two different cell types, wherein a first cell type is localized to or within a first spatially defined region of the construct having a first extracellular matrix protein microstructure and a second cell type is localized to or within a second spatially defined region of the construct having a second extracellular matrix protein microstructure.

33. The engineered tissue construct of claim 30, wherein the construct is selected from the group consisting of a muscular tissue construct, a vascular tissue construct, an esophageal tissue construct, an intestinal tissue construct, a rectal tissue construct, an ureteral tissue construct, a cartilaginous tissue construct, a cardiac tissue construct, a liver tissue construct, a bladder tissue construct, a kidney tissue construct, a pancreatic tissue construct, a skeletal tissue construct, a filamentous/ligament tissue construct, a lung tissue construct, a neural tissue construct, a bone tissue construct, and a skin tissue construct.

34. The engineered tissue construct of claim 30, wherein the construct is vascularized.

35. A method of treating a subject in need of tissue repair or tissue replacement, said method comprising:
selecting a subject in need of tissue repair or tissue replacement;
providing the engineered tissue construct of claim 30; and
implanting the engineered tissue construct into the selected subject.

36. The monolithic tissue scaffold of claim 25, wherein said tissue scaffold is produced by a method comprising:
providing a biological composition comprising at least one soluble unpolymerized extracellular matrix protein;
adjusting the conditions of the biological composition to effect extracellular matrix protein polymerization; and
exposing the biological composition to one or more ultrasound wave fields during extracellular matrix protein polymerization under conditions effective to control extracellular matrix protein microstructure within one or more regions of the biological composition.
